(19) United States
(12) Patent Application Publication
(10) Pub. No.: US 2004/0097717 A1
(43) Pub. Date: May 20, 2004

(54) Control of aerial branching

(76) Inventors: Ottoline Leyser, York (GB); Jonathan Booker, York (GB); Karim Sorefan, Norwich (GB)

Correspondence Address: Gray Cary Ware & Freidenrich, 4365 Executive Drive Suite 1100, San Diego, CA, 92121-2133, US

(21) Appl. No.: 10/239,818

(22) PCT Filed: Mar. 23, 2001
(86) PCT No.: PCT/GB01/01308

(30) Foreign Application Priority Data
Mar. 24, 2000 (GB) 0007291.8

(51) Int. Cl. C07H 21/02; C07H 21/04

(52) U.S. Cl. 536/23.1

(57) ABSTRACT

A plant nucleic acid sequence is provided which encodes a protein involved in the synthesis of abscisic acid. The plant nucleic acid sequence, and proteins encoded thereby, are useful in the regulation of aerial branching in plants.

This invention relates to plant nucleic acid and promoter sequences and proteins. The sequences and proteins are useful in the control of aerial branching in plants.

The pattern of shoot branching and the growth characteristics of lateral shoots determine to a large extent the growth habit of plants. In seed plants, shoot branching is initiated by the formation of lateral meristems in the leaf axil (Steeves and Sussex, 1989). In the axils of developing leaf primordia, distinct groups of meristematic cells, which are in direct continuity with the shoot apical meristem, can be recognised. In Arabidopsis, auxiliary meristems can be detected only much later after the transition of the shoot apical meristem to reproductive development (Gubic and Bleecker, 1996). In some plant species, the apical meristem of the primary shoot remains active throughout the life of the plant and continues to initiate the formation of lateral organs (for example, Arabidopsis and Antirrhinum). In other plant species, the primary apical men stem at some point of development undergoes the transition to floral development or it aborts. Further development of axillary buds into side shoots is controlled by the main shoot apex, which very often exerts an inhibitory influence on apical buds. This phenomenon is known as apical dominance. Apical dominance can be defined as the condition in which there is a concentration of resources in the main stem of the plant and a corresponding suppression of axillary branches.

A mutant defective in axillary meristem initiation has been identified in tomato. This mutant is the lateral suppresser (LS) mutant and leads to the absence of side shoots in the vegetative green phase (Schumacher et al 1999). In addition, LS plants have a defect in petal development leading to the absence of certain flower organs and a consequent reduction in male and female sterility thereby preventing the use of this mutation in conventional breeding programs.

Plants exhibit different developmental patterns of aerial branching ranging from species where apical dominance is high and there is little branch formation to species where apical dominance is low and the plant is very bushy. The domestication of crop plants is often involved in an increase in apical dominance. A striking example of this is seen in domesticated maize which exhibits a profound increase in apical dominance compared with its wild ancestor teosinte (Iltis, 1983). The reason for this increase in apical dominance is due to a twofold increase in expression of the TB1 gene, isolated by (Doebly et al., 1997). However, tb1 maize mutants, in addition to exhibiting increased branching, have no female inflorescences (ears). It has been suggested that TB1 both acts to suppress the growth of axillary organs and enable the formation of female inflorescences.

The control of aerial branching is of agronomic interest in several areas. Branching patterns influence the effectiveness of light harvest and thus plant yield. Branching patterns influence plant competitivity either by directing resources to overgrow other plants or by creating a dense canopy to prevent other plants growing. Moreover, branching patterns influence the synchronicity of flowering non-synchronous formation of floral branches leads to seed yield losses as either more mature seed is shed or some seed is immature at harvest. Branching patterns may also influence the number of flowers per inflorescence influencing for example, fruit size and yield.

For gardening purposes, highly branched plants are desirable. Branched plants are useful as hedges and the appearance of the lateral branching can add to the aesthetic value of garden plants. However, in most cases highly branched plants are undesirable. Lateral branching in plants inevitably restricts the room available for growth of adjacent plants. This is a particular problem where plants are grown for timber as fewer plants will mean lower wood yield. In addition, branching in plants channels resources from the main stem into the branches which is undesirable in situations where main stem yield is important for timber. A further problem associated with highly branched plants is the knotting of the branches. Knotting will hinder the logging process as well as reducing the yield of wood and as such is a major economic problem in the timber producing industry. The present invention provides a solution to these problems.

According to a first aspect of the invention there is provided a nucleic acid selected from

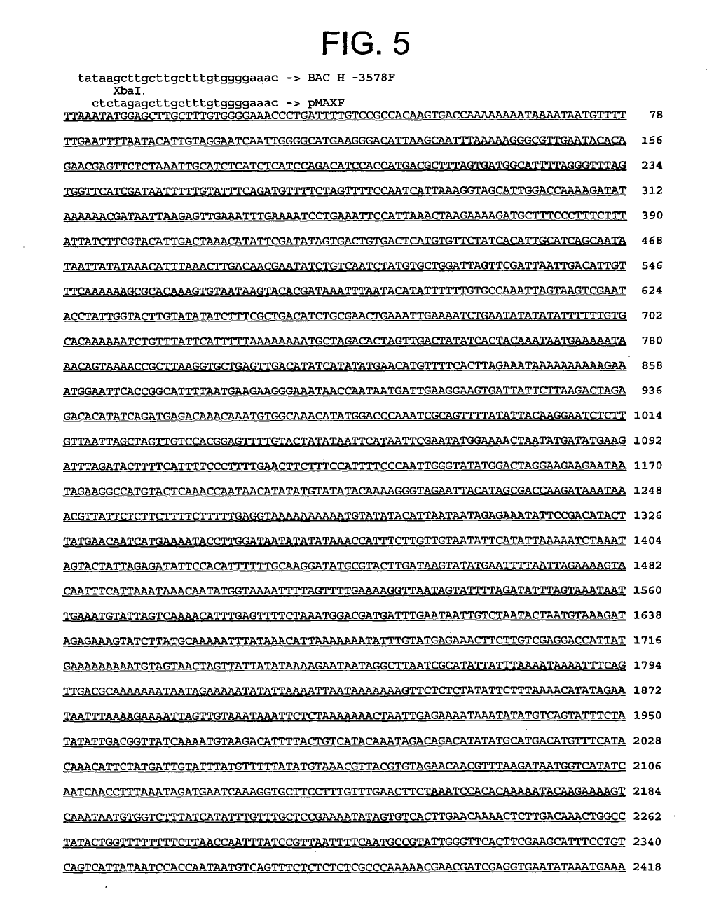

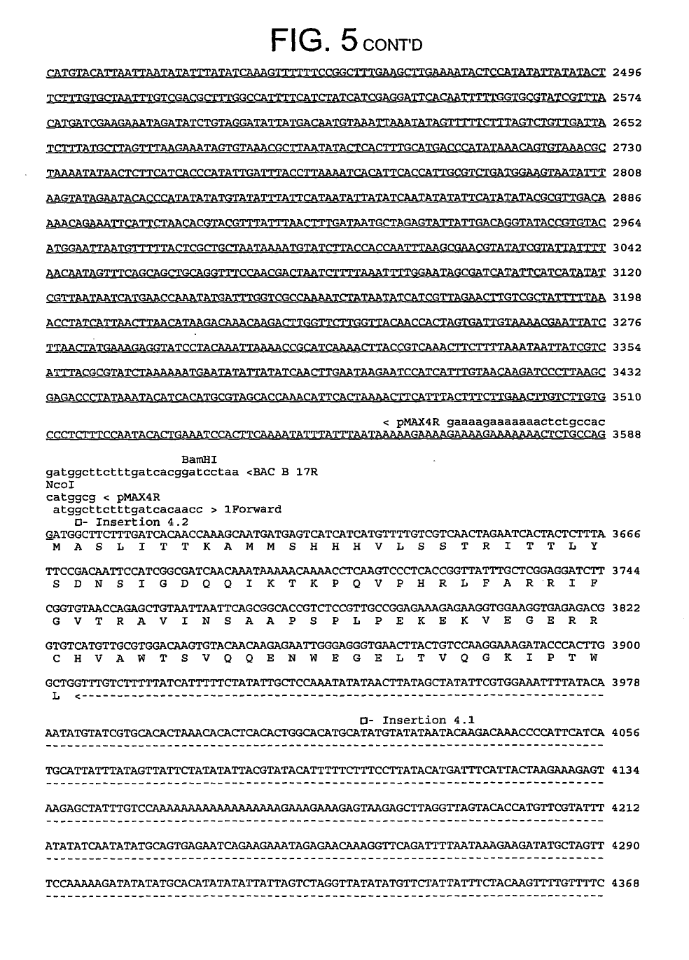

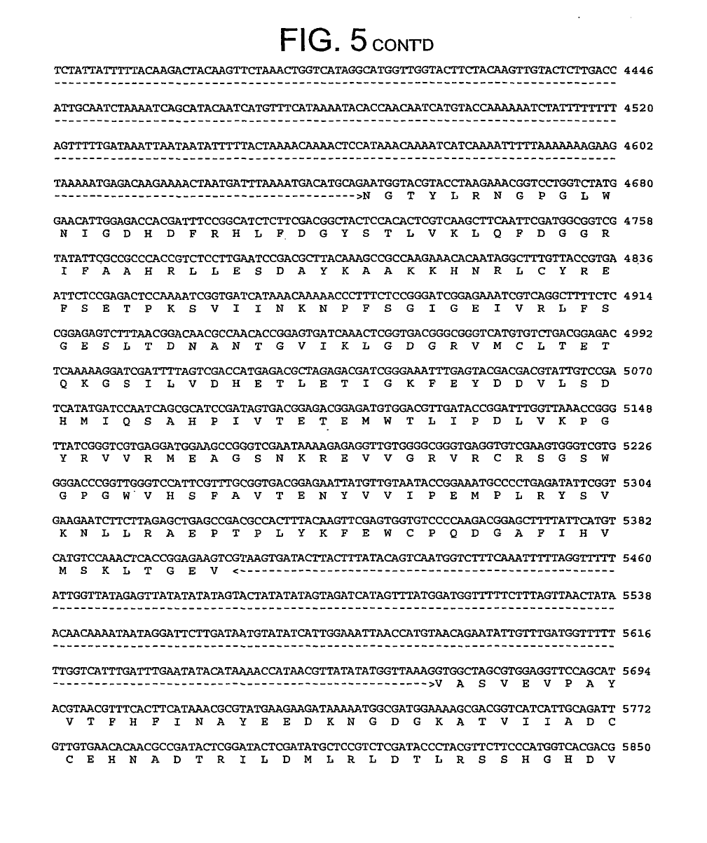

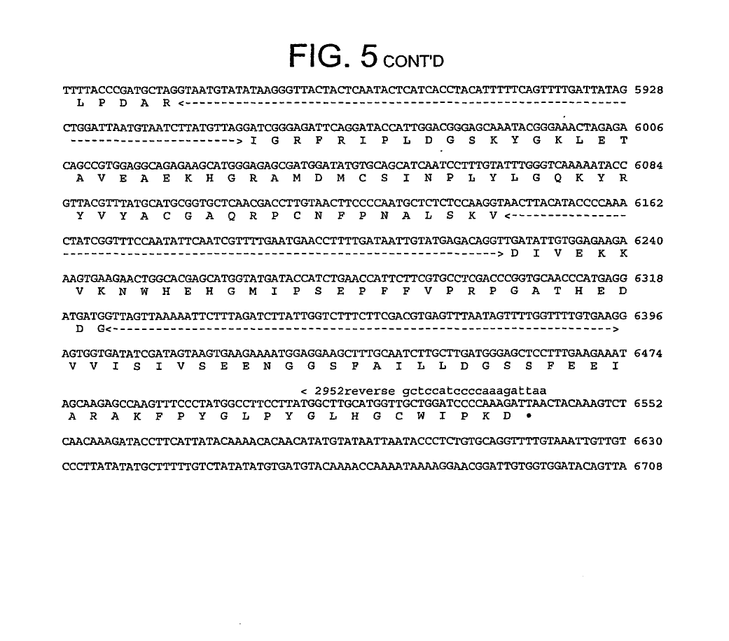

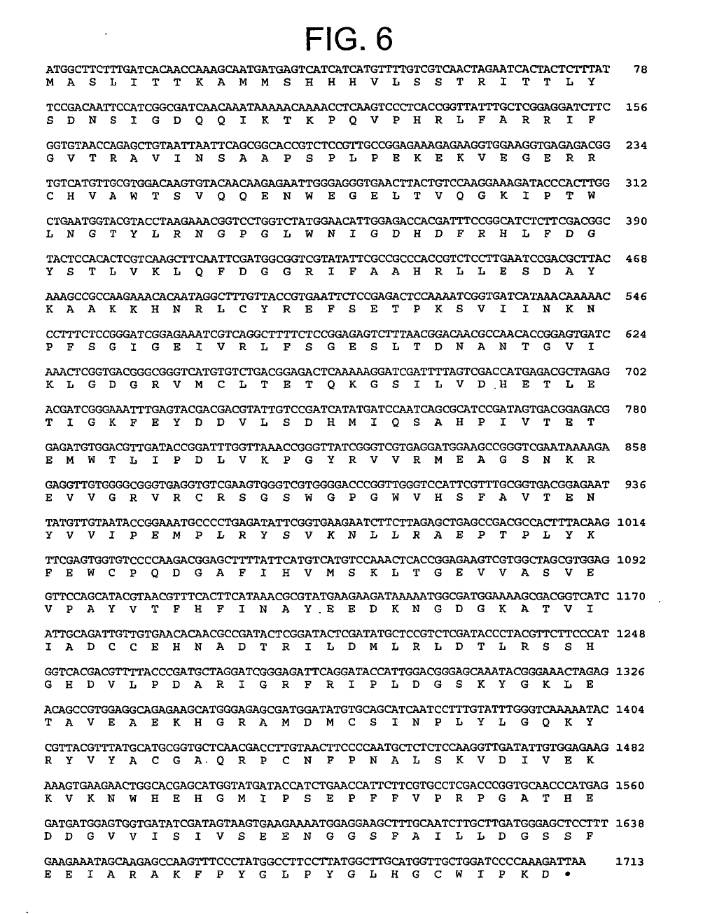

(i) a DNA sequence comprising all or part of the DNA sequence of FIG. 5 or FIG. 6 or its complementary strand;

(ii) nucleic acid sequences hybridising to the DNA sequence of FIG. 5 or FIG. 6 or its complementary strand under stringent conditions;

(iii) nucleic acid sequences which would hybridise to the DNA sequence of FIG. 5 or FIG. 6 or its complementary strand but for the degeneracy of the genetic code.

As used herein “part of the DNA sequence” includes fragments of the DNA sequence, for example of at least 15, 20, 30, 40 or 60 nucleotides in length.

Fragments of the nucleic acid and/or nucleic acid sequences, for example of at least 15, 20, 30, 40 or 60 nucleotides in length, are also within the scope of the invention.

Suitable stringent conditions include salt solutions of approximately 0.9 molar at temperatures of from 35° C. to 65° C. More particularly, stringent hybridisation conditions include 6×SSC, 5× Denhardt's solution, 0.5% SDS, 0.5% tetrasodium pyrophosphate and 50 mcg/rnl denatured herring spern DNA; washing may be for 2×30 minutes at 65° C. in 1×SSC, 0.1% SDS and 1×30 minutes in 0.2×SSC, 0.1% SDS at 65° C. Stringent conditions may encompass “highly stringent conditions” or “moderately stringent conditions”. Highly stringent conditions means hybridisation to DNA bound to a solid support in 0.5M NaHPO₄, 7% SDS, 1 nM EDTA at 65° C. and washing in 0.1×SSC/0.1% SDS at 68° C. (Ausubel et al (1989)). In some circumstances, less stringent hybridisation conditions may be required. Moderately stringent conditions means washing in 0.2×SSC/0.1% SDS at 42° C. (Ausubel et al (1989)). Hybridisation conditions can also be rendered more stringent by the addition of increasing amount of formamide, to destabilise the hybrid duplex. Thus, particular hybridsation conditions can be readily manipulated, and will generally be selected according to the desired results.

Nucleic acid sequences within the scope of the first aspect of the invention will generally encode a protein involved in the synthesis of abscisic acid (ABA). In this text, the term “involved in the synthesis of ABA” means any nucleic acid optionally encoding any protein which is on, or involved in, the ABA synthetic pathway or any other protein or nucleic acid which results in changes in the expression of a gene involved in ABA synthesis. The proteins of the invention which are involved in the synthesis of ABA may include one or more of isomerase, dioxygenase, epoxidase, oxidase, oxygenase, hydrolase, cyclase, de-epoxidase, desaturase or synthase.

The term “protein” in this text means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues and derivatives of a protein wherein the fragment, analogue or derivative retains essentially the same biological activity or function as a reference protein.

The fragment, derivative or analogue of the protein may be (i) one in which one or more of the amino acid residues are substituted with a conserved or non-con served amino acid residue (preferably, a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half life of the polypeptide (for example, polyethylene glycol), or (iv) one in which the additional amino acids are fused to the mature polypeptide, such as a leader or secretory sequence which is employed for purification of the polypeptide. Such fragments, derivatives and analogues are deemed to be within the scope of those skilled in the art from the teachings herein.

Particularly preferred are variants, analogues, derivatives and fragments having the amino acid sequence of the protein in which several e.g. 5 to 10, 1 to 5, 1 to 3, 2, 1 or no amino acid residues are substituted, deleted or added in any combination. Especially preferred among these are silent substitutions, additions and deletions, which do not alter the properties and activities of the protein of the present invention. Also especially preferred in this regard are conservative substitutions.

An example of a variant of the present invention is a fusion protein as defined above, apart from the substitution of one or more amino acids with one or more other amino acids. The skilled person is aware that various amino acids have similar properties. One or more such amino acids of a substance can often be substituted by one or more other such amino acids without eliminating a desired activity of that substance.

Thus the amino acids glycine, alanine, valine, leucine and isoleucine can often be substituted for one another (amino acids having aliphatic side chains). Of these possible substitutions it is preferred that glycine and alanine are used to substitute for one another. (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, argmine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi-conservative” amino acid substitutions.

Amino acid deletions or insertions may also be made relative to the amino acid sequence for the fusion protein referred to above. Thus, for example, amino acids which do not have a substantial effect on the activity of the polypeptide, or at least which do not eliminate such activity, may be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

Amino acid insertions relative to the sequence of the fusion protein above can also be made. This may be done to alter the properties of a substance of the present invention (e.g. to assist in identification, purification or expression, as explained above in relation to fusion proteins).

Amino acid changes relative to the sequence given in a) above can be made using any suitable technique e.g. by using site-directed mutagenesis.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

A protein according to the invention may have additional N-terminal and/or C-terminal amino acid sequences. Such sequences can be provided for various reasons, for example, glycosylation.

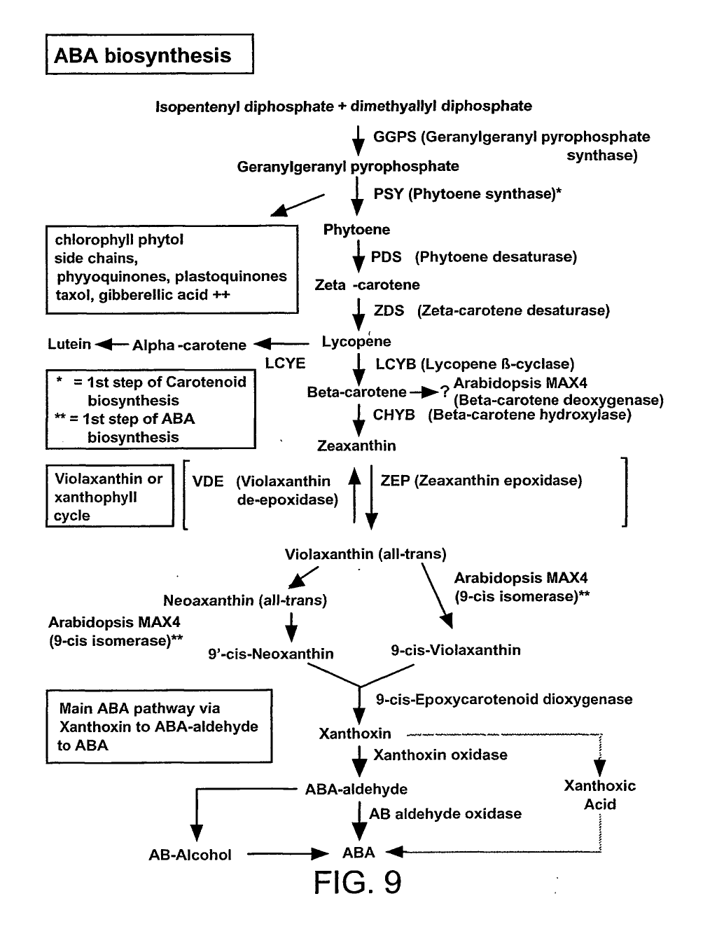

The nucleic acid of the present invention preferably encodes proteins which catalyse one or more of the reactions involved in the synthesis of ABA, or effect one or more of the steps involved in the synthesis of ABA, as shown in FIG. 9. Preferably, the nucleic acid of the present invention encodes a protein which is an isomerase enzyme or a dioxygenase enzyme, in particular an enzyme which catalyses one or more dioxygenase or isomerase steps, for example the steps from all trans violaxanthin to 9 cis neoxanthin or from beta-carotene to 9 cis neoxanthin and 9 cis violaxanthin.

The nucleic acid of the first aspect of the present invention may encode a protein involved in the regulation of aerial branching in plants. In this text, the term “involved in the regulation of aerial branching” means any nucleic acid (preferably) encoding any protein which has an effect on aerial branching, in particular a protein/nucleic acid involved in controlling the outgrowth of aerial lateral branches.

Typically, the nucleic acid of the present invention encodes a protein which regulates the growth of lateral branches, in particular the growth of axillary branches.

The nucleic acid or protein of the present invention which is involved in the regulation of aerial branching may alter the branching of floral inflorescence in plants.

Furthermore, the nucleic acid sequence or protein of the present invention which involved in the regulation of aerial branching may alter root branching in plants.

The nucleic acid of the first aspect of the invention may be a nucleic acid which is naturally expressed in the, for example, aerial parts, or vasculature, of plants, for example, the meristem, leaf, bud, branches, leaf nodes. Such a nucleic acid will most accurately reflect nucleic acid naturally expressed in plants.

Typically, the plant may be a member of any plant family. Preferably the plant is a member of the Brassicaceae family, for example, members of the Brassica genus such as Brassica napus and Arabidopsis thaliana.

The nucleic acid of the first aspect of the present invention typically comprises the sequence set out in FIG. 5 or FIG. 6 or a fragment thereof which may be at least 15 nucleotides in length.

Expression of the nucleic acid of the present invention in plants may decrease the degree of aerial branching. Decreased aerial branching can be achieved by over-expressing the nucleic acid of the present invention from its own promoter, or other suitable promoter.

The nucleic acid of the first aspect of the invention may be antisense. As understood by the person skilled in the art, introducing the coding region of a gene in the reverse orientation to that found in nature (antisense) can result in the downregulation of the gene and hence the production of less or none of the gene product. The transcribed antisense DNA is capable of binding to and destroying the function of the sense RNA of the sequence normally found in the cell, thereby disrupting function. Antisense nucleic acid may be constitutively expressed, but it is preferably limited to expression in those parts of the plant in which the naturally occurring nucleic acid is expressed. Expression of the antisense to nucleic acid according to the first aspect of the invention, in plants increases the degree of aerial branching. Downregulation can be achieved by other methods known in the art, such as expression of full sense or partial sense transcripts homologous to nucleic acid according to the first aspect of the invention. Alternatively, downregulation may be achieved by the expression of ribosomes that are designed to cleave transcripts encoded by the nucleic acid of the first aspect of the invention.

The nucleic acid of the first aspect of the invention preferably includes a promoter or other regulatory sequence which controls expression of the nucleic acid. The person skilled in the art will know that it may not be necessary to utilise the whole promoter or other regulatory sequence. Only the minimum essential regulatory elements may be required, the essential requirement being to retain the tissue and/or temporal specificity. Preferably, the promoter or other regulatory sequence which controls expression of a nucleic acid according to the first aspect of the invention comprises all or part of the underlined sequence as set out in FIG. 5. Elements in the 5′untranslated region of FIG. 5 may contribute to the promoter and for this reason have been included in the underlined sequence. Promoters which control expression of a nucleic acid of the first aspect of the invention may be the naturally occurring promoter (its own promoter). Typically, expression of the nucleic acid of the first aspect of the invention under the control of the naturally occurring promoter in plants suppresses aerial branching.

All preferred features of the first aspect of the invention as described above also apply to the second and subsequent aspects of the invention mutatis mutandis.

A second aspect of the invention provides a nucleic acid sequence encoding the amino acid sequence of FIG. 6.

The nucleic acid of the first and second aspects of the invention may be isolated or recombinant or may be in substantially pure form.

By “isolated” is meant a polynucleotide sequence which has been purified to a level sufficient to allow allelic discrimination. For example, an isolated sequence will be substantially free of any other DNA or protein product. Such isolated sequences may be obtained by PCR amplification, cloning techniques, or synthesis on a synthesiser. By “recombinant” is meant polynucleotides which have been recombined by the hand of man.

The third aspect of the invention relates to a promoter sequence selected from

(i) a DNA sequence comprising all or part of the DNA sequence underlined in FIG. 5 or its complementary strand; and

(ii) nucleic acid sequences hybridising to the DNA sequence underlined in FIG. 5 or its complementary strand under stringent conditions.

The promoter may be provided in combination with the nucleic acid of the first or second aspect of the invention. Alternatively, the promoter may be provided in combination with another gene of interest, for example, one or more genes involved in sucrose metabolism, starch synthesis, hormone synthesis, perception, signalling, or the production of transporter proteins (for hormones, sugars, nutrients, nucleotides, anions, cations), RNAases, cellulases, proteases, glucanases, antibacterial agents or waterproofing agents. The promoter may be axil- or vasculature-specific. The vasculature may be of leaves, stems, sepals, siliques or roots. The vasculature may be phloem or xylem Alternatively, the promoter may be leaf specific.

The promoter of the third aspect of the invention may be isolated or recombinant or may be in substantially pure form.

The present invention also provides RNA encoded by nucleic acid according to the first or second aspect of the invention. Moreover, the present invention provides RNA encoded by the promoter sequence according to the third aspect of the invention.

A protein which is the expression product of a nucleic acid according to the first or second aspect of the invention, or an RNA encoded by this nucleic acid, is provided by the invention. The protein may be isolated or recombinant or may be in substantially pure form. An antibody capable of binding to the protein is also within the scope of the present invention.

The nucleic acid according to the first or second aspect of the invention and the promoter sequence according to the third aspect of the invention may be in the form of a vector. The vector may be a plasmid, cosmid or phage. Vectors frequently include one or more expressed markers which enable selection of cells transfected, or transformed, with them and preferably, to enable a selection of cells, containing vectors incorporating heterologous DNA. A suitable start and stop signal would generally be present and if the vector is intended for expression, sufficient regulatory sequences to drive expression will be present. Nucleic acid and promoter sequences according to the invention are preferably for expression in plant cells. Microbial host expression and vectors not including regulatory sequences are useful as cloning vectors.

A fourth aspect of the invention relates to a cell comprising nucleic acid according to the first or second aspect of the invention or promoter sequence according to the third aspect of the invention. The cell may be termed as a “host” which is useful for manipulation of the nucleic acid or promoter, including cloning. Alternatively, the cell may be a cell in which to obtain expression of the nucleic acid or promoter, most preferably a plant cell. The nucleic acid or promoter can be incorporated into cells by standard techniques known in the art. Preferably, nucleic acid is transformed into plant cells using a disarmed Ti plasmid vector and carried an agrobacterium by procedures known in the art, for example, as described in EP-A-0116718 and EP-A-0270822. Foreign nucleic acid can alternatively be introduced directly into plant cells using an electrical discharged apparatus or by any other method that provides for the stable incorporation of the nucleic acid into the cell. Nucleic acid according to the first or second aspect of the invention preferably contains a second “marker” gene that enables identification of the nucleic acid. This is most commonly used to distinguish the transformed plant cells containing the foreign nucleic acid from other plant cells that do not contain the foreign nucleic acid. Examples of such marker genes include antibiotic resistance, herbicide resistance and glucoronidase (GUS) expression. Expression of the marker gene is preferably controlled by a second promoter, which is preferably not the promoter of the third aspect of the invention, which allows expression of the marker gene in cells other than axil cells. Preferably the cell is from Brassica napus, pea, sunflower, maize or wheat.

A fifth aspect of the invention includes a process for obtaining a cell comprising nucleic acid according to the first or second aspect of the invention or promoter sequence according to the third aspect of the invention. The process involves introducing the nucleic acid or promoter sequence into a suitable cell and optionally growing or culturing said cell.

A sixth aspect of the invention provides a plant or a part thereof comprising a cell according to the fifth aspect of the invention. A whole plant can be regenerated from the single transformed plant cell by procedures well known in the art. The invention also provides for propagating material or a seed comprising a cell according to the fifth aspect of the invention. The invention also relates to any plant or part thereof including propagating material or a seed derived from any aspect of the invention. The sixth aspect of the invention also includes a process for obtaining a plant or plant part, the process comprising obtaining a cell according to the fifth aspect of the invention or plant material according to the sixth aspect of the invention and growth thereof.

A seventh aspect of the invention provides a protein which

(i) comprises the amino acid sequence shown in FIG. 5; or

(ii) has one or more amino acid deletions, insertions or substitutions relative to a protein as defined in (i) above, and has at least 40% amino acid sequence identity therewith; or

(iii) a fragment of a protein as defined in (i) or (ii) above which is at least 10 amino acids long.

The percentage amino acid identity can be determined using the default parameters of the GAP computer program, version 6.0, described by Deveraux et al 1984 and available from the University of Wisconsin Genetics Computer Group (UWGCG). The GAP program utilises the alignment method of Needleman and Wunsch 1970 and revised by Smith and Waterman 1981. More preferably the protein has at least 45% identity to the amino acid sequence of FIG. 5, through 50%, 55%, 60%, 65%, 70%, 75%, 80%, 85%, 90% and 95% identity using the default parameters.

The protein of the seventh aspect of the invention may be a biologically active protein or a protein which is antigenic. The protein of the seventh aspect of the invention is typically full-length as in FIG. 6. Alternatively, the protein may be a fragment of at least 10, 15, 20, 30 or 60 amino acids in length and which is biologically active and/or antigenic.

The present invention provides nucleic acid which encodes a protein of the seventh aspect of the invention.

The protein of the seventh aspect of the invention may be isolated or recombinant or may be in substantially pure form. The protein preferably comprises a transit peptide sequence, for example, a chloroplast transit peptide sequence.

The eighth aspect of the invention provides a process for regulating/controlling aerial branching in a plant or in a part thereof, the process comprising obtaining a plant or a part thereof according to the sixth aspect of the invention. The process of aerial branching can be regulated and/or controlled by increasing or decreasing the expression of nucleic acid according to the first or second aspect of the invention. Increased or decreased expression can easily be influenced by the person skilled in the art using technology well known. This includes increasing the number of copies of nucleic acid according to the invention in a plant or plant part thereof or increasing expression levels of copies of the nucleic acid present in particular parts or regions of the plant. Increased expression levels of copies of the nucleic acid of the present invention may take place in the leaf axils or vasculature of the plant due to expression being regulated by the promoter sequence according to the third aspect of the invention. Preferably, increased expression levels of copies of the nucleic acid of the present invention takes place in the vasculature of the plant.

The process according to the eighth aspect of the invention also provides a process for the synthesis of abscisic acid. The process of abscisic acid synthesis can be regulated and/or controlled by increasing or decreasing the expression of nucleic acid according to the first or second aspect of the invention. Abscisic acid synthesis in the plant, for example, in the leaf axil or vascular regions, may directly or indirectly regulate aerial branching in the plant.

The process according to the eighth aspect of the invention includes obtaining a plant cell according to the fifth aspect of the invention or part of a plant according to the sixth aspect of the invention and deriving a plant therefrom. Alternatively, the process may comprise obtaining propagating material or a seed according to the sixth aspect of the invention and deriving a plant therefrom.

The process of the eighth aspect of the invention may take place in the vasculature or axil of a plant, for example, the leaf axil. Preferably, the process of the eighth aspect of the invention takes place in the vasculature of a plant.

A ninth aspect of the invention provides for the use of nucleic acid according to the first to eighth aspects of the invention in the regulation/control of aerial branching in plants.

The tenth aspect of the invention provides for the use of nucleic acid according to the first to ninth aspects of the invention for the synthesis of abscisic acid. Preferably, the use according to the tenth aspect of the invention, regulates a plants response to water stress. In this context water stress comprises drought stress and/or flooding.

The tenth aspect of the invention further provides for the use of a nucleic acid according to the first to ninth aspects of the invention in the regulation/control of preharvest sprouting. Preferably, the use according to the tenth aspect of the invention is in the embryo and/or endosperm of plants. Further uses of the nucleic acid according to the first to ninth aspects of the invention include the regulation of plant dormancy and/or the regulation of drought tolerance.

The eleventh aspect of the invention provides for the use of nucleic acid according to the first or second aspect of the invention as a probe. Such a probe can be used in techniques well known in the art to identify the presence of identical or homologous nucleic acid sequences from any source, preferably a plant source. The eleventh aspect of the invention also provides nucleic acid identified by use of the nucleic acid from the first or second aspect of the invention as a probe.

A twelfth aspect of the invention provides for the use of nucleic acid according to the first or second aspect of the invention in the production of a cell, tissue, plant or part thereof, or propagating material.

A thirteenth aspect of the invention provides for nucleic acid comprising one or more of the primer sequences as shown in the examples. Such nucleic acid sequences are preferably used as primers in a PCR polymerase chain reaction) process in order to amplify nucleic acid sequences.

A fourteenth aspect of the invention provides for the use of a protein according to the seventh aspect of the invention as a probe. In this context the probe is a means to identifying entities which interact with the protein, for example, other proteins. A protein according to the seventh aspect of the invention can be used with a probe to directly look for interactions with other proteins, for example, purified protein can be used to look for complex formation with other plant proteins. Alternatively, the protein of the seventh aspect of the invention can be used to prepare an antibody to the protein. This antibody can then be used to identify protein complexes and to purify the complexes.

A fifteenth aspect of the invention provides a method for the regulation of aerial branching in plants, the method comprising the steps of

(i) transforming the plant with nucleic acid as claimed in claim 1;

(ii) expression of said nucleic acid in a plant under the control of a promoter.

Typically the promoter is the naturally occurring promoter. The promoter may be the promoter of the third aspect of the invention which controls expression of nucleic acid in, for example, the vasculature or leaf axils. Preferably, the promoter is the promoter of the third aspect of the invention which controls expression of nucleic the vasculature. Promoters which are not the naturally occurring promoter and which may be used in accordance with the fifteenth aspect of the invention include embryo and/or endosperm specific promoters, bud-specific promoters, leaf-specific promoters or any other suitable promoter from a plant species. Alternatively, the promoter may a be a synthetic promoter sequence.

A sixteenth aspect of the invention provides a method for regulating the synthesis of abscisic acid in plants the method comprising the steps of

(i) transforming the plant with nucleic acid as claimed in claim 1;

(ii) expression of said nucleic acid in a plant under the control of a promoter.

All preferred features of the fifteenth aspect of the invention also apply to the sixteenth.

The methods of the fifteenth or sixteenth aspect of the invention may comprise the steps of

(i) transforming the plant with antisense to nucleic acid according to the first or second aspect of the invention;

(ii) expression of said antisense in a plant under the control of a promoter.

The invention is described by reference to the Figures as follows:

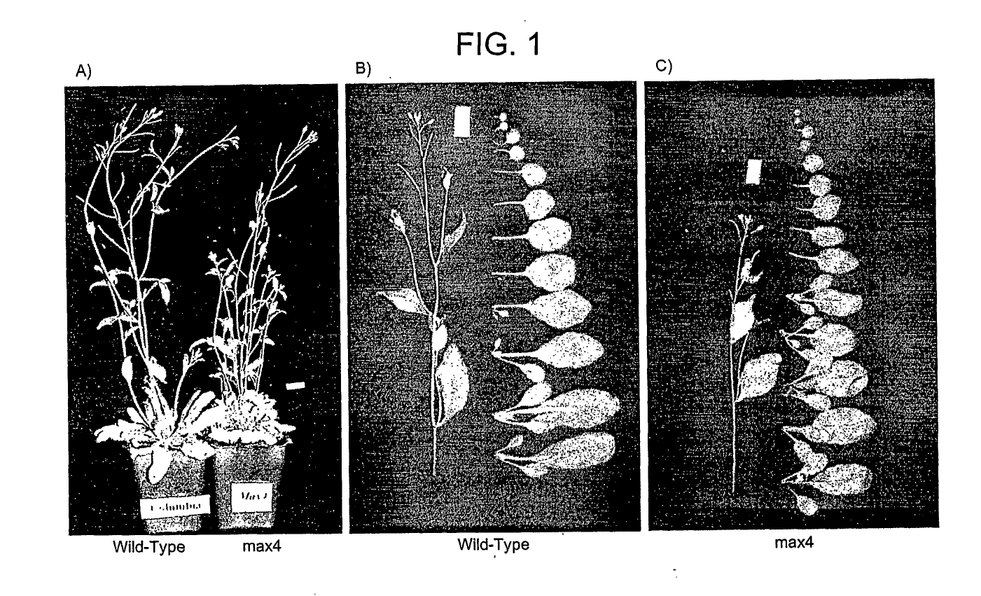

FIG. 1—a) Growth habit of the homozygous max4.1 mutant compared to wild-type three weeks after germination. Disection of wild-type (b) and Max4.1 mutant plants to show greater extent of axillary bud development in Max4.1.

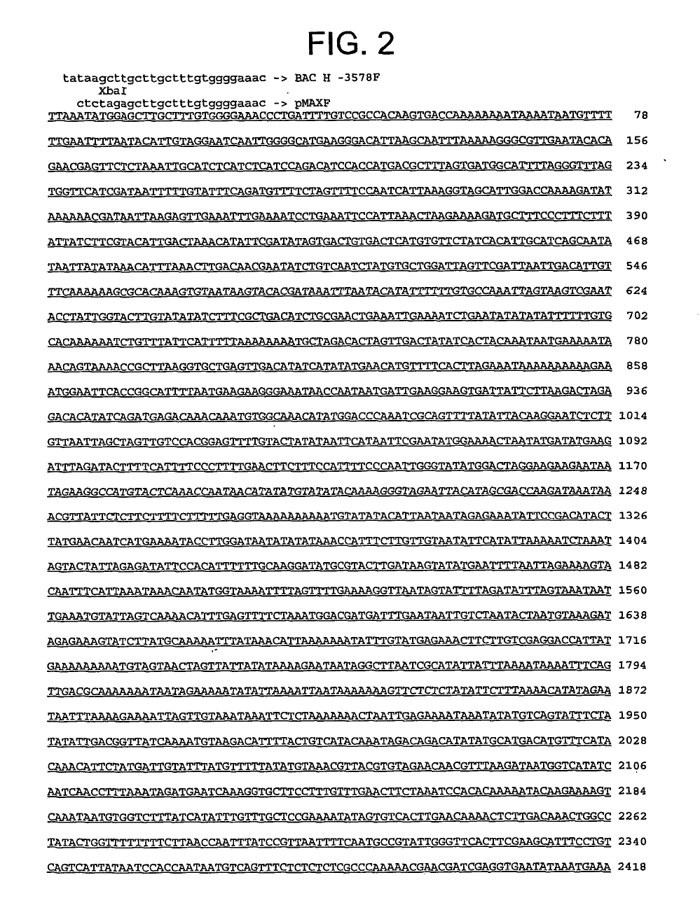

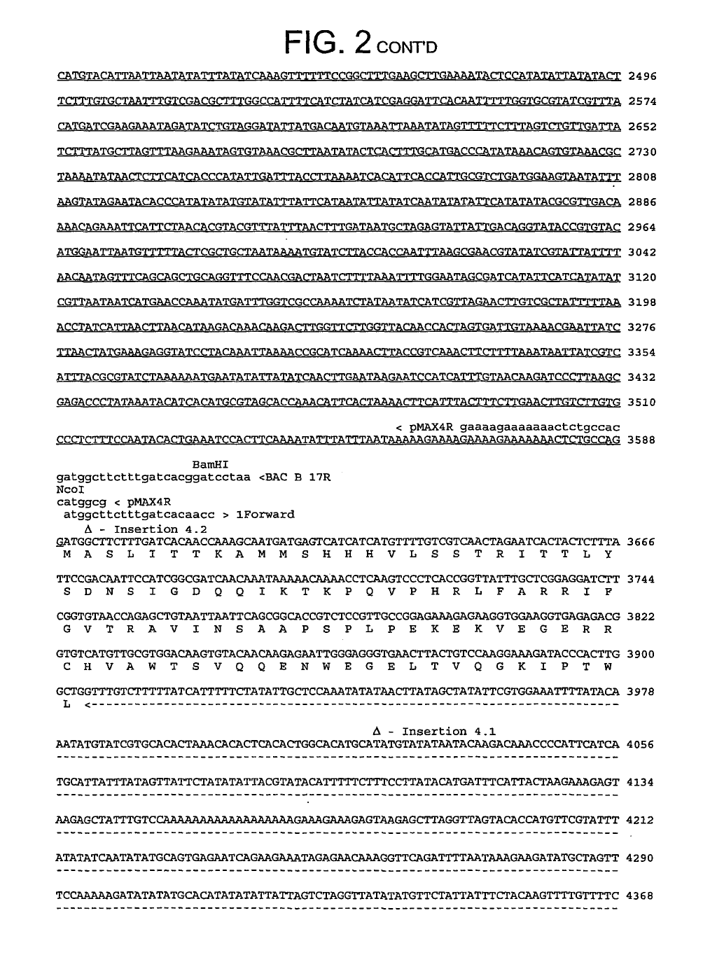

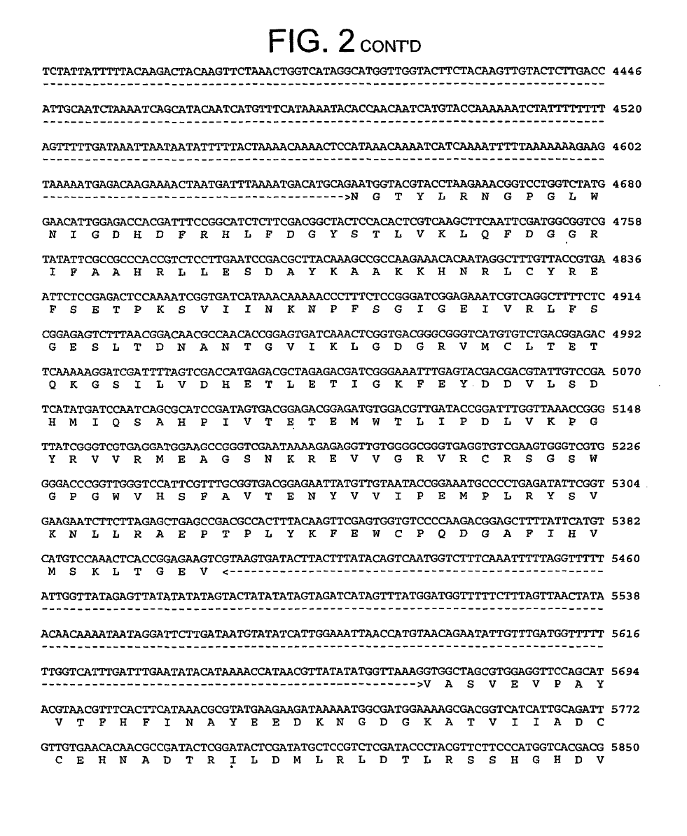

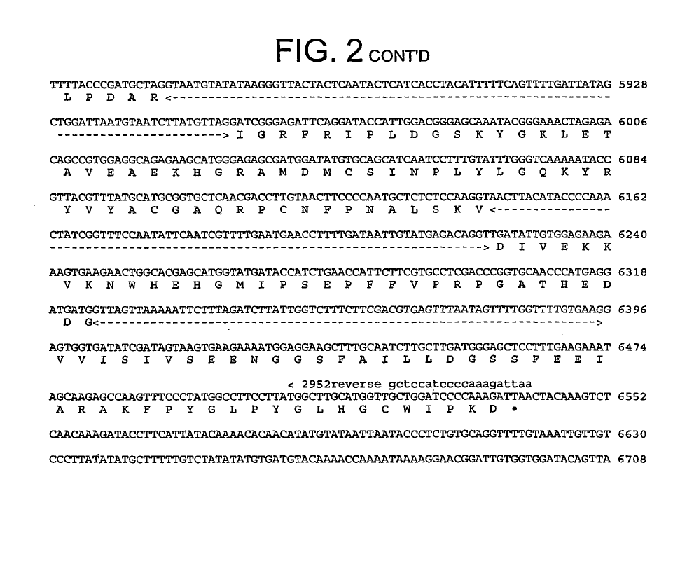

FIG. 2—Sequence of the MAX4 gene present on BAC AL049915. The positions of the En insertions in max4.1 and max4.2 are indicated above the DNA sequence. En inserts in front of the A-marked nucleotide. The putative MAX4 protein sequence is indicated below the DNA sequence. Primers used to PCR the MAX4 cDNA and MAX4 promoter fragments are indicated.

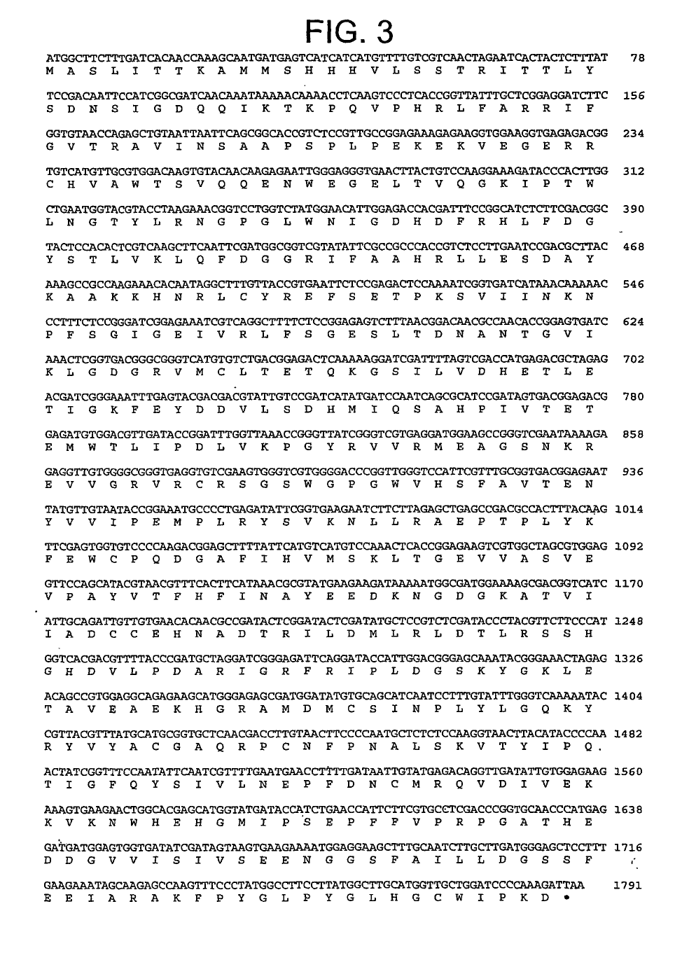

FIG. 3—Sequence of the MAx4 cDNA. The putative MAX4 protein sequence is indicated below the DNA sequence.

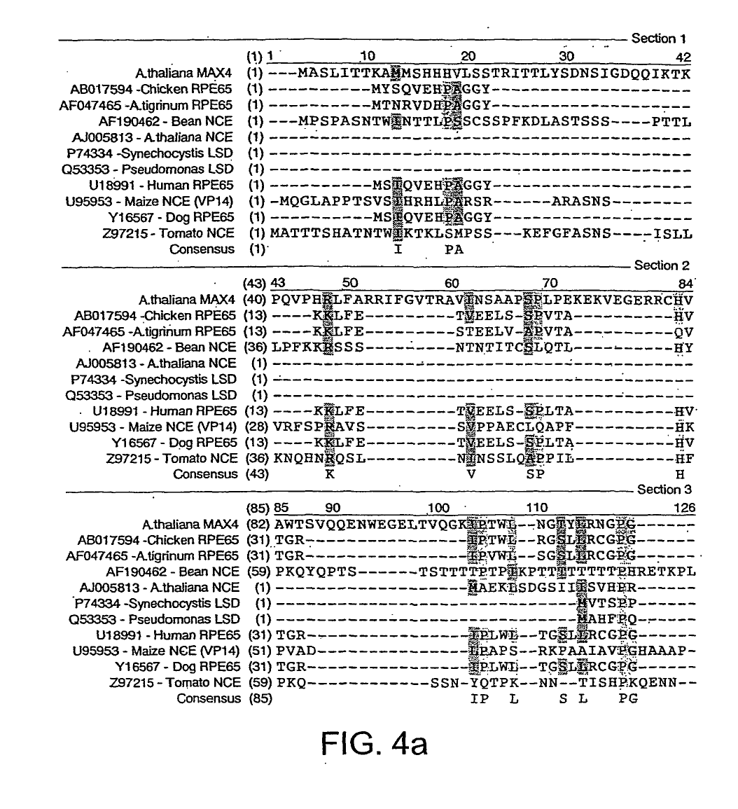

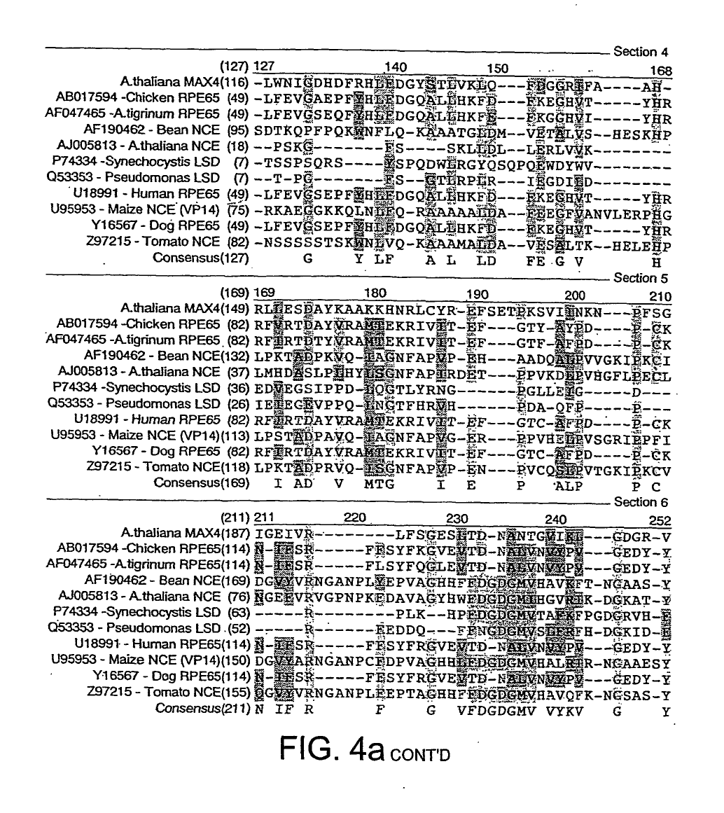

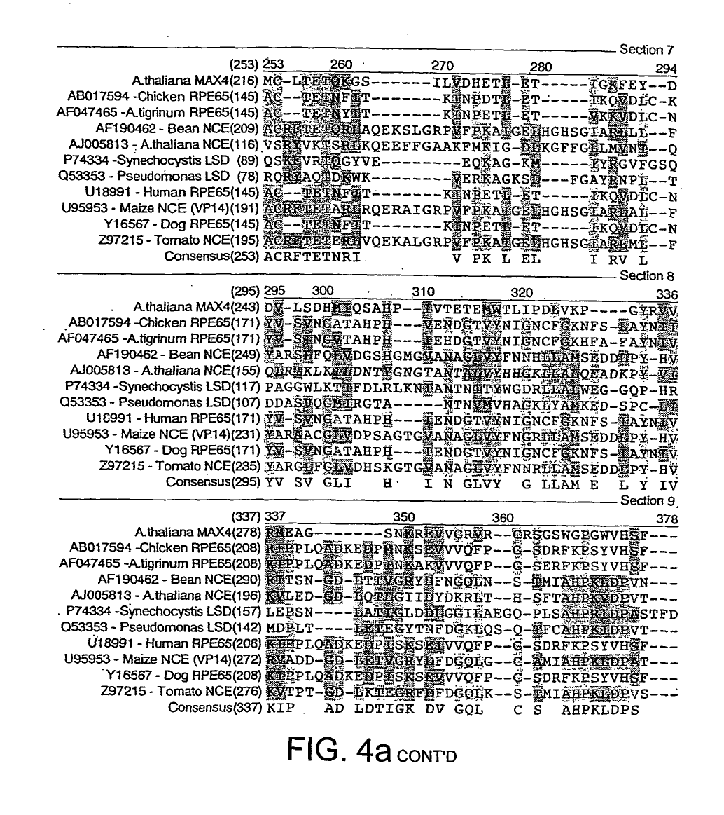

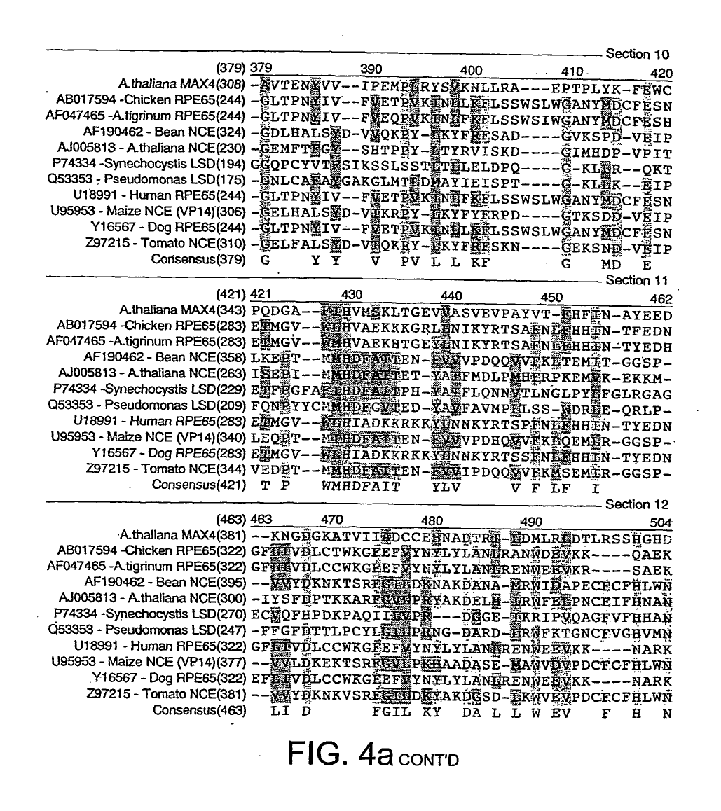

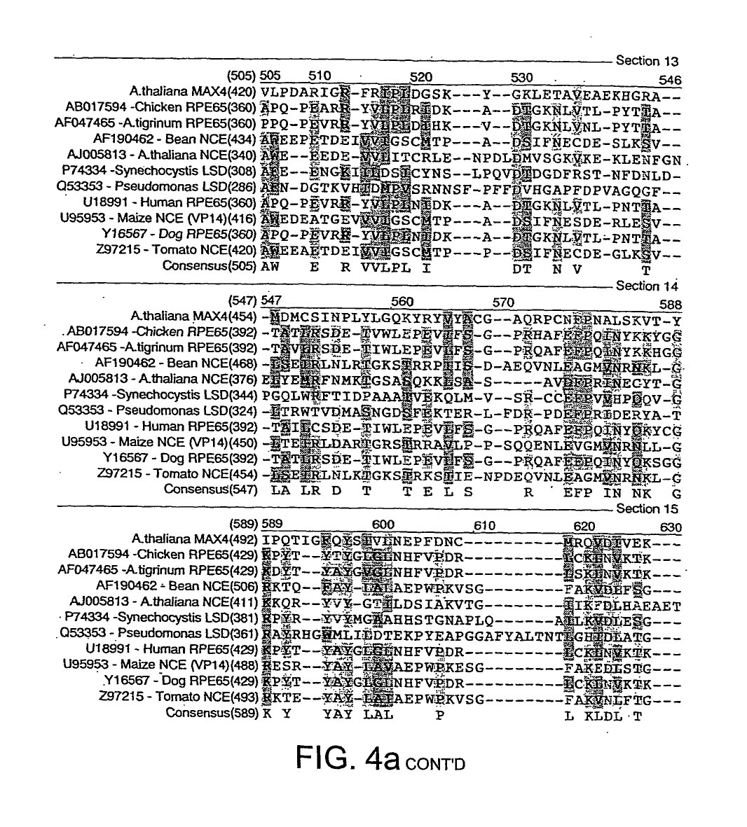

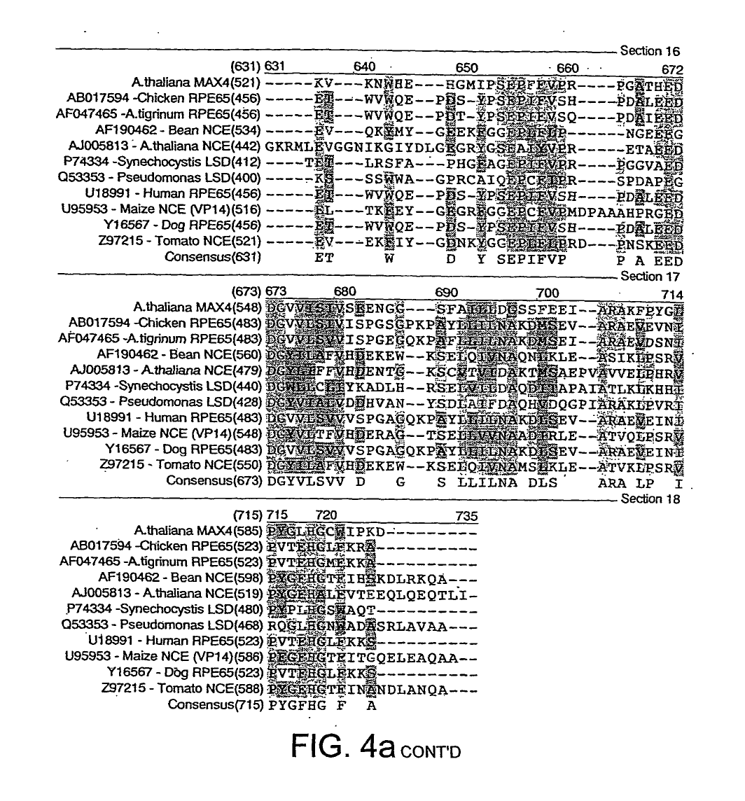

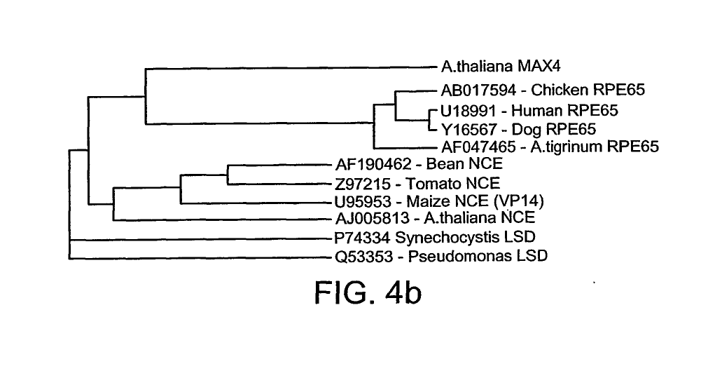

FIG. 4—a) Alignment of the MAX4 putative protein sequences shown in FIG. 3 and FIG. 3; accession numbers are indicated. b) Dendrogram constructed from the alignment.

FIG. 5—Sequence of the MAX4 gene present on BAC AL049915. The positions of the En insertions in max4.1 nd max4.2 are indicated above the DNA sequence. En inserts in front of the □-marked nucleotide. The putative MAX4 protein sequence is indicated below the DNA sequence (note: the sequence of the MAX4 gene is identical to the gene sequence shown in FIG. 2; the putative protein sequence is, however, shorter than the sequence shown in FIG. 2). Primers used to PCR the MAX4 cDNA and MAX4 promoter fragments are indicated.

FIG. 6—Sequence of the MAX4 cDNA. The putative MAX4 protein sequence is indicated below the DNA sequence (note: the sequence of the MAX4 cDNA is identical to the sequence shown in FIG. 3 except that it is shorter as nucleotides 1467 to 1545 inclusive are absent from the sequence. Consequently, the putative MAX4 protein sequence is shorter than the deduced sequence shown in FIG. 3).

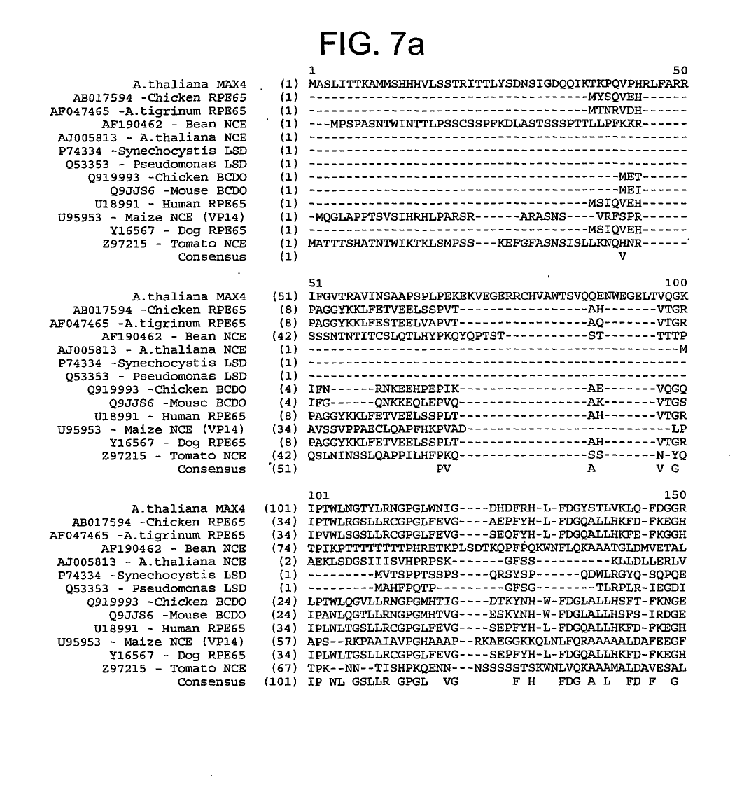

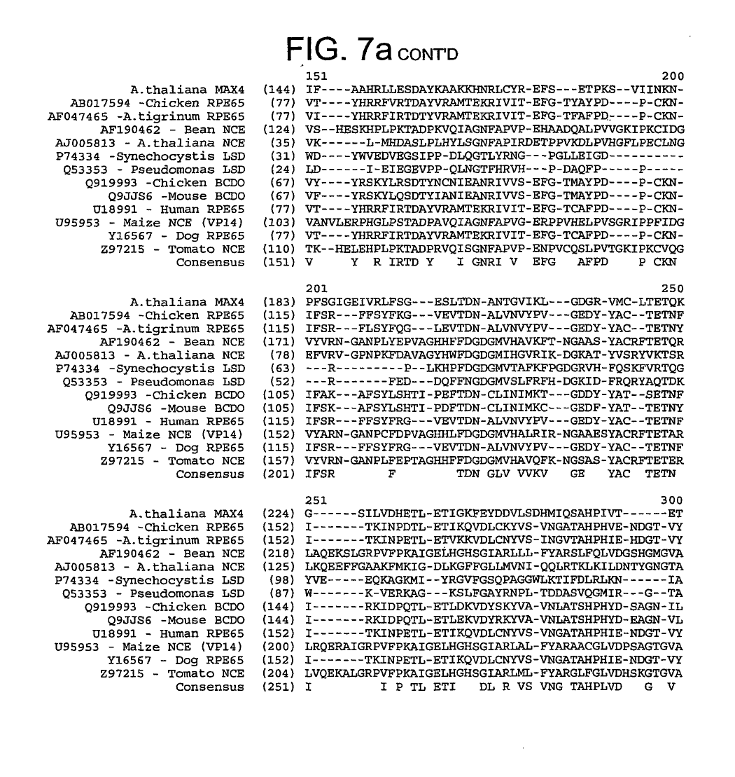

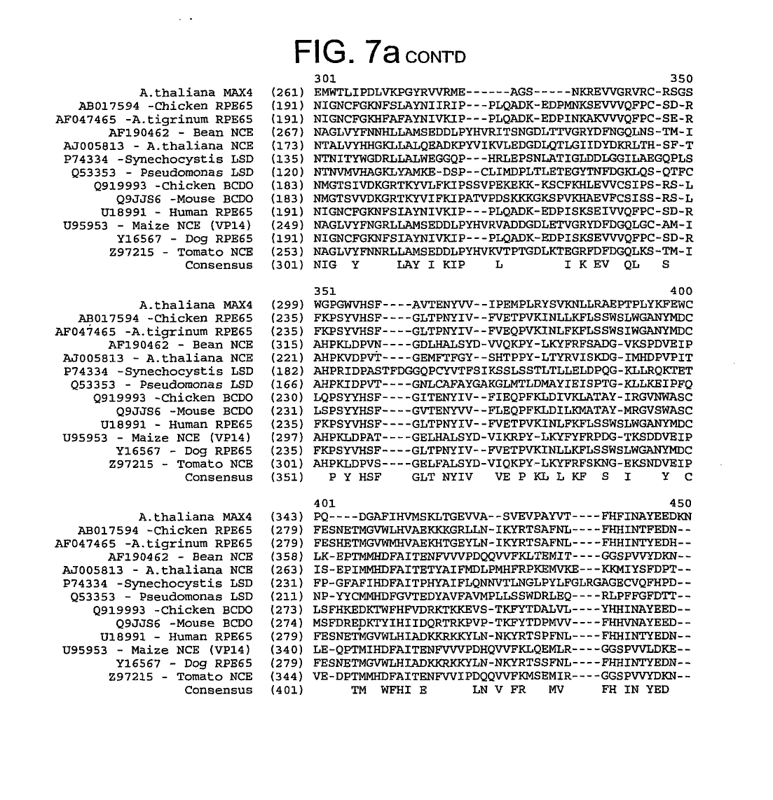

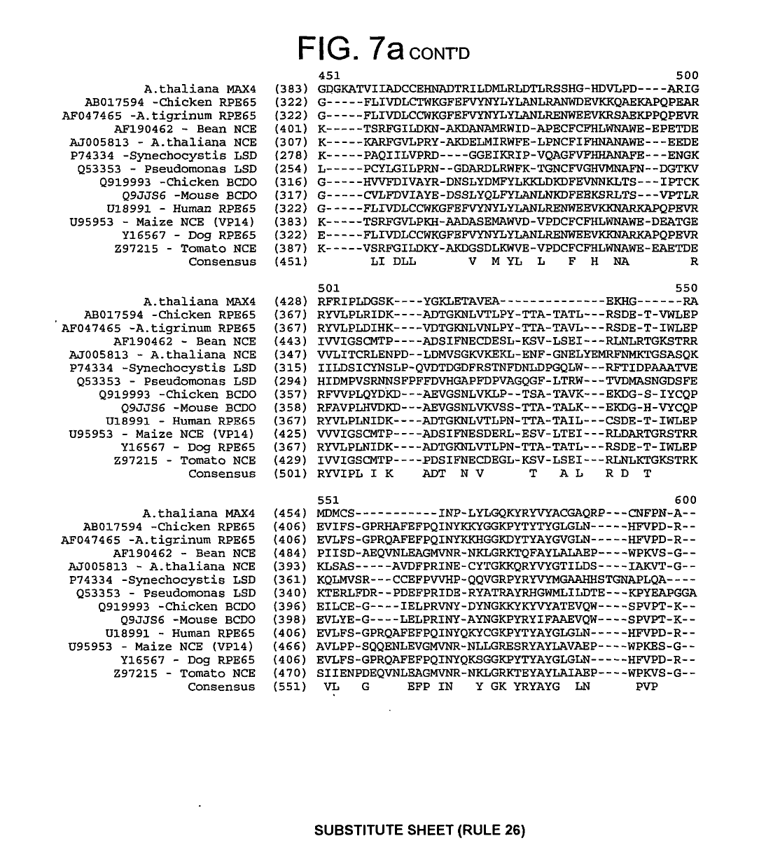

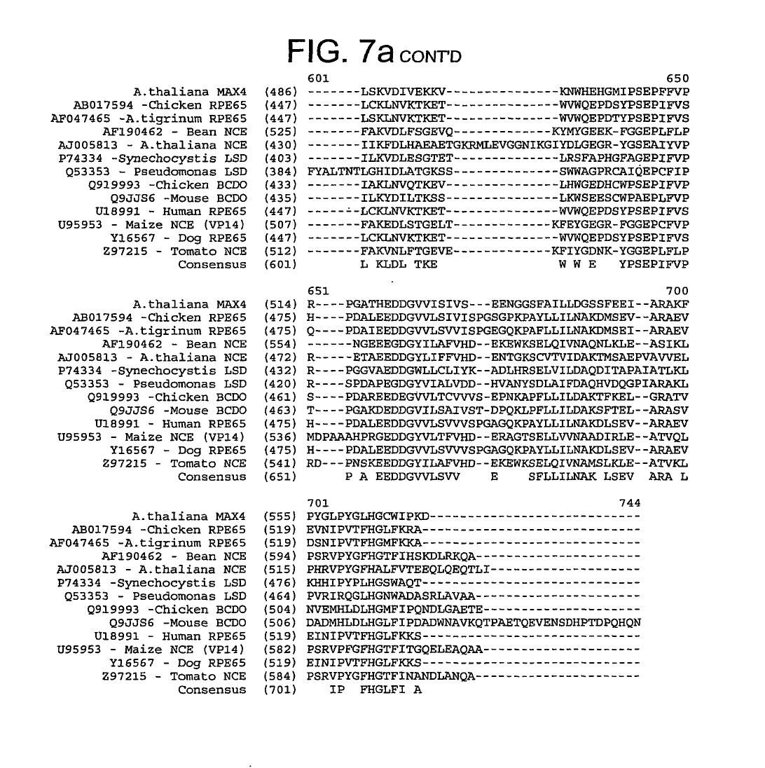

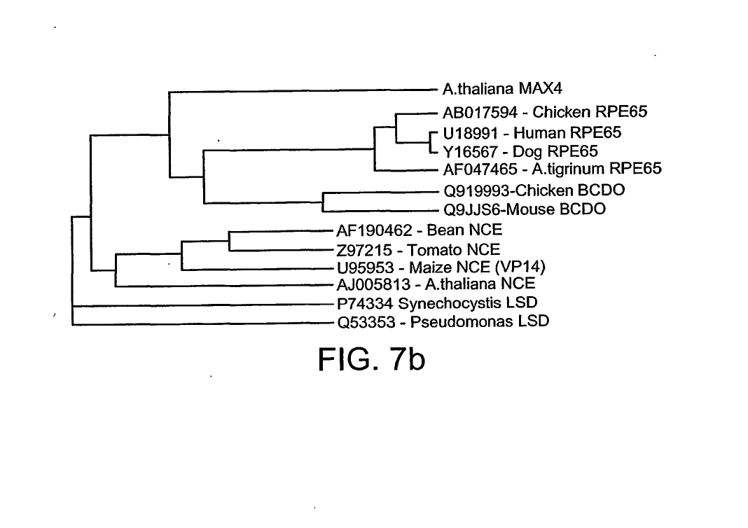

FIG. 7—a) Alignment of the MAX4 putative protein sequence shown in FIG. 5 and FIG. 6; accession numbers are indicated. b) Dendrogram constructed from the alignment.

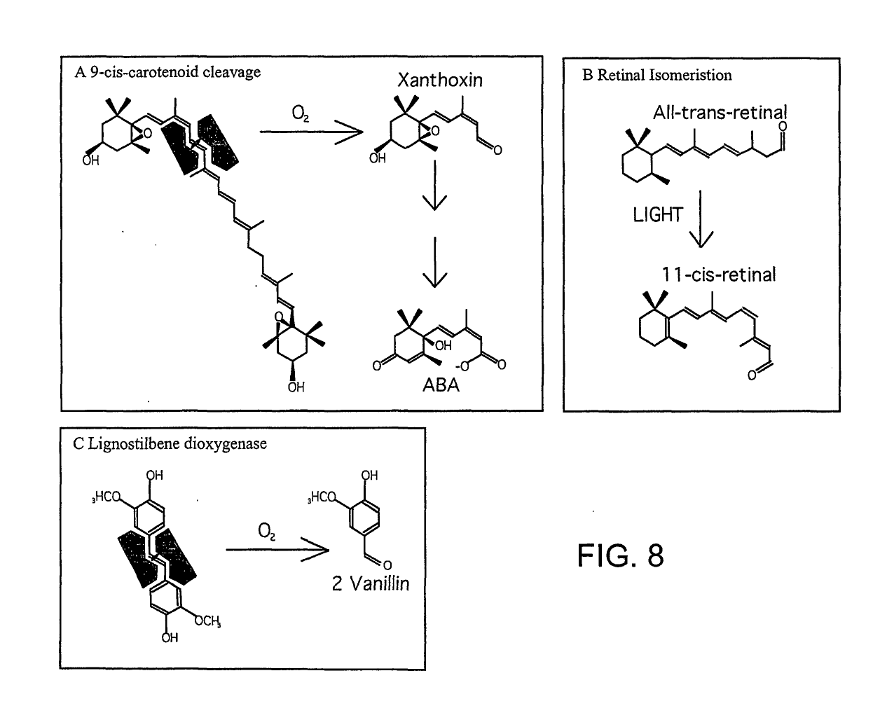

FIG. 8—Proposed reactions catalysed by (a) VP14, (b) RPE65 and (c) Lignostilbene dioxygenase. Wavy lines indicate sites of cleavage.

FIG. 9—Scheme showing the biosynthesis of ABA.

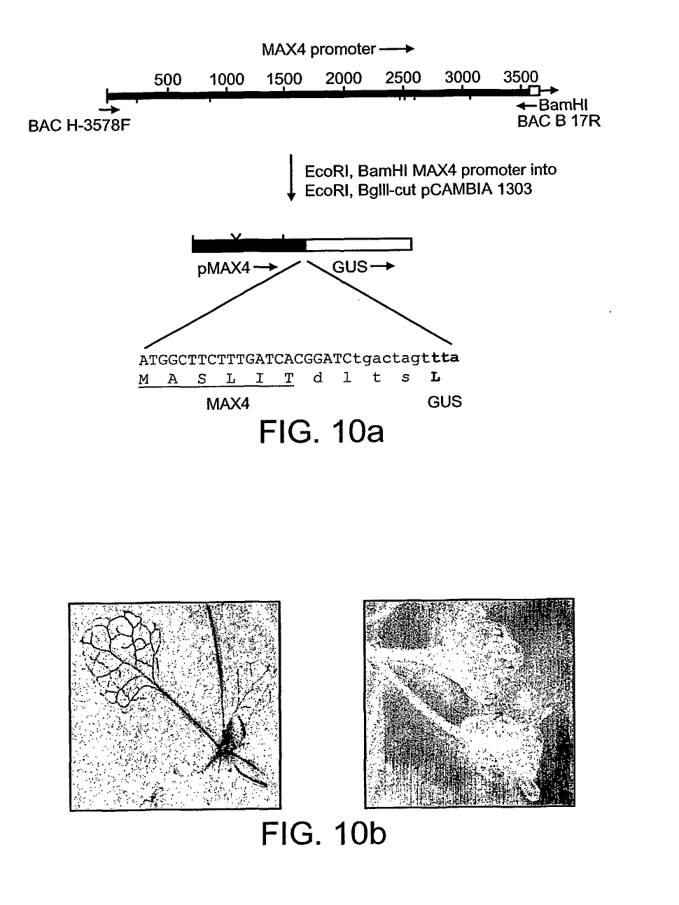

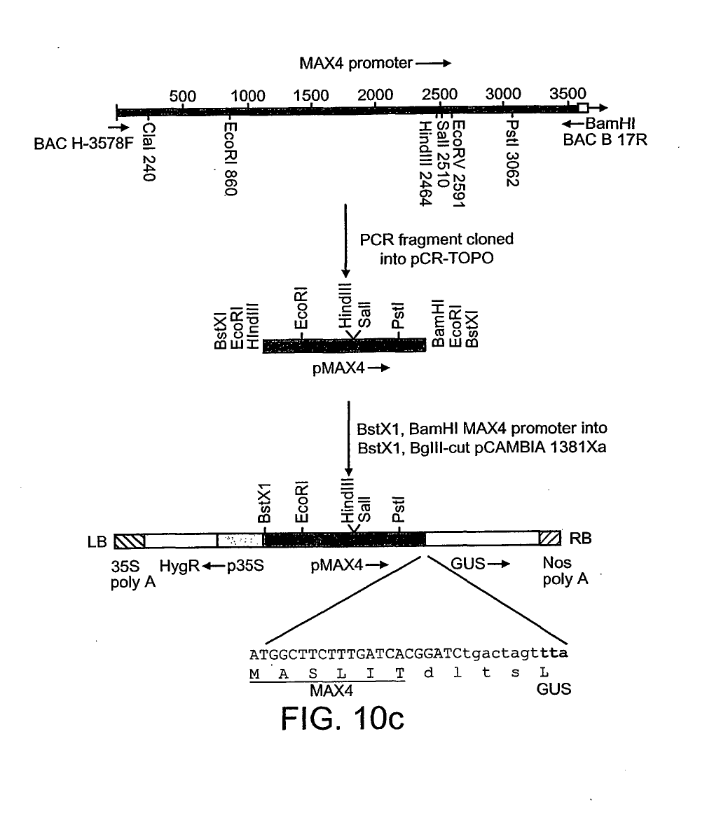

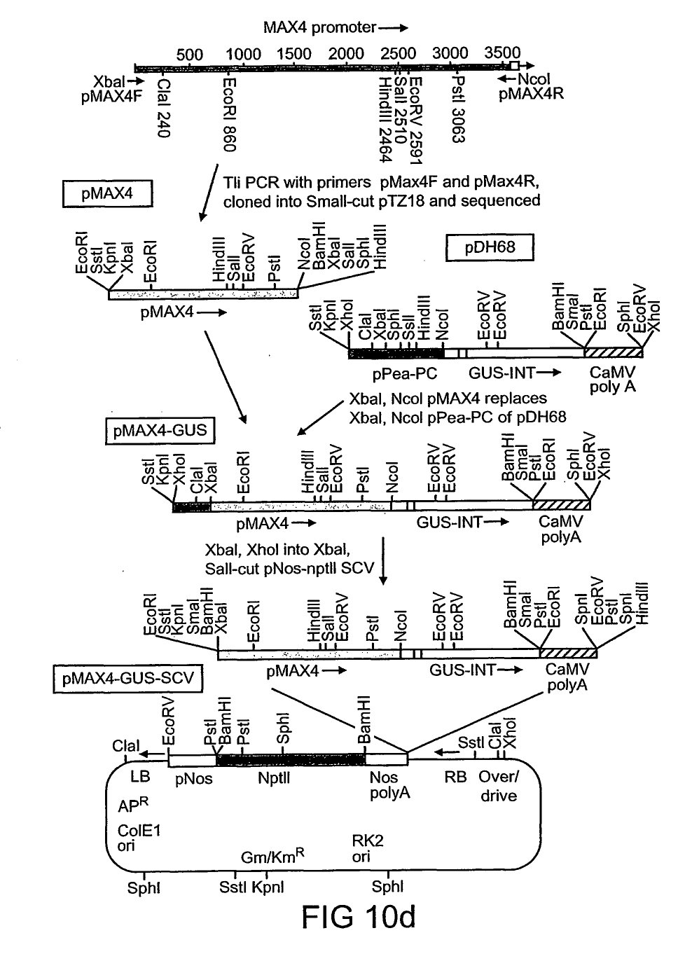

FIG. 10—Schematic diagram showing the construction of pMAX4-GUS fusions. a.) simplified schematic diagram showing the construction of a pMAX4-GUS-CAMBIA fusion and b.) promoter activity in transgenic A. thaliana; GUS expression is shown in a representative A. thaliana transformant. c.) schematic diagram showing the construction of the pMAX4-GUS-CAMBIA fusion used in preliminary studies d.) construction of pMAX4-GUS-SCV.

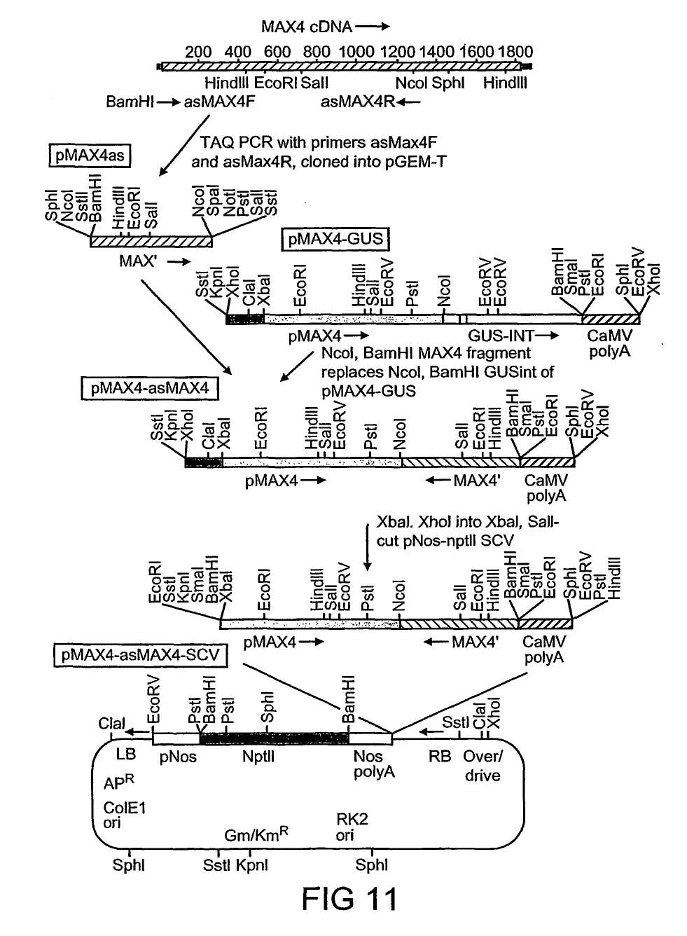

FIG. 11—Schematic diagram showing the construction of pMAX4-asMAX4-SCV.

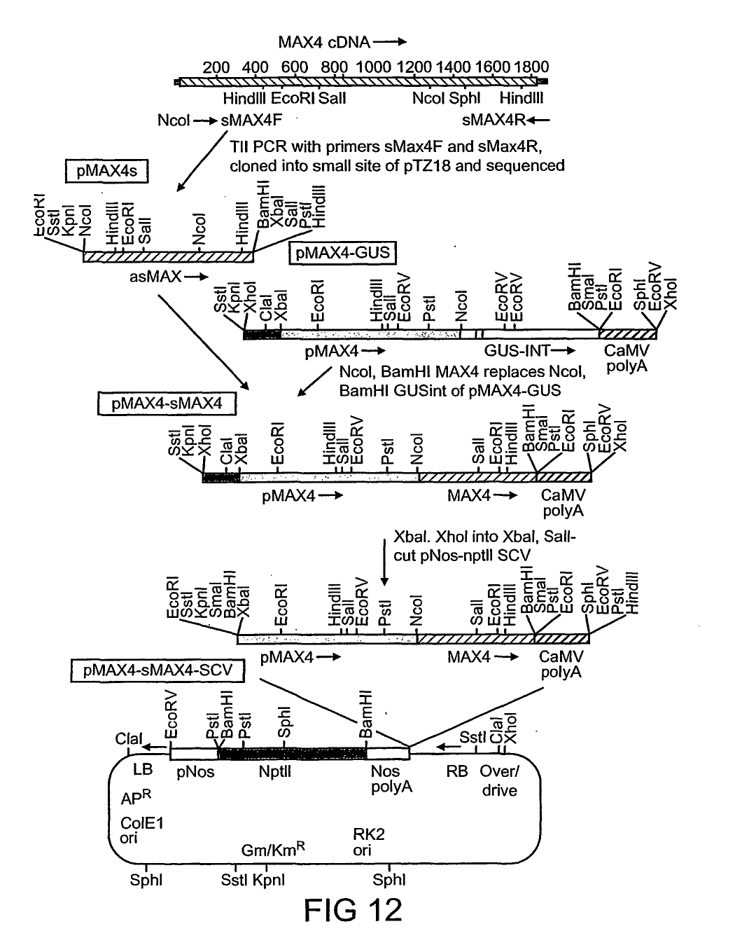

FIG. 12—Schematic diagram showing the construction of pMAX4-sMAX4-SCV.

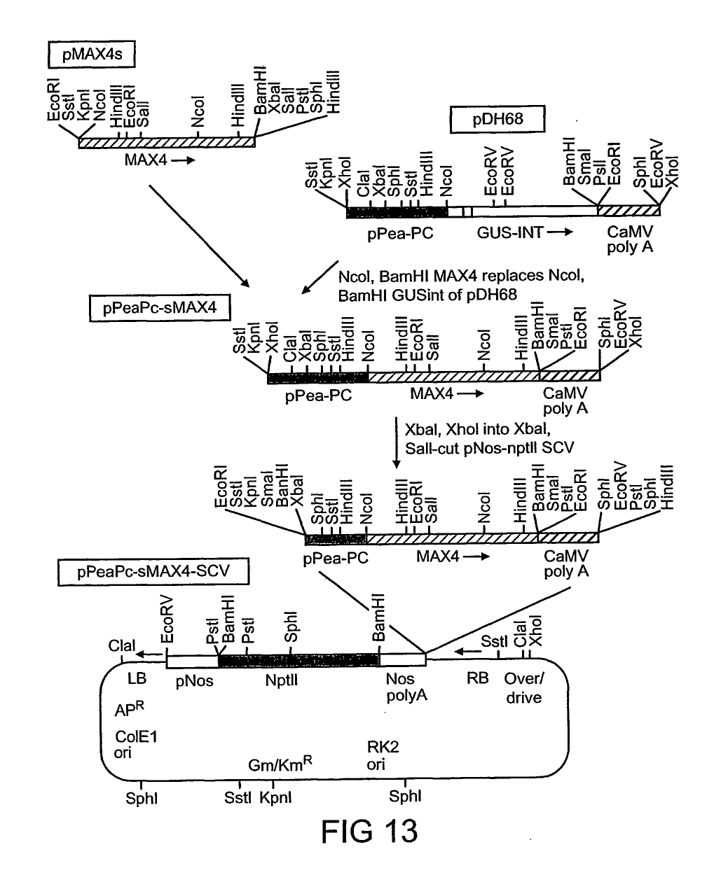

FIG. 13—Schematic diagram showing the construction of pPeaPC-sMAX4-SCV.

The present invention is now described with reference to the following non-limiting examples.

### EXAMPLE 1

### Isolation a max4 Mutant Line and Cloning of the MAX4 Gene

Screens of mutagenised Arabidopsis thaliana populations for plants with a bushy, reduced apical dominance phenotype isolated plants which had a more axillaries (max) phenotype. In these max mutants the bushy phenotype is due to a lack of repression of axillary bud outgrowth so that all the axillary buds elongate, even the ones close to the vegetative meristem that would not elongate (FIG. 1). Complementation studies revealed that the mutations fell into 4 groups max1 to max4. In order to clone the MAX genes max mutants were isolated from an En mutagenised population (SLAT population) (J. Jones, Sainsbury Lab, JIC). Two mutants were found to be allellic to max4; max4.1 and max4.2. These max4 plants were bushy, dwarfed and had rounded leaves and shorter petioles (FIG. 1). Otherwise the these max4 plants appear phenotypically normal and fertile.

Analysis of the F1 and F2 generations, resulting from a backcross of a homozygous max4 lines to Columbia-0 WT, indicated that the max4 phenotypes segregate as a single recessive mutation. It was determined by Southern analysis that max4.1 and max4.2 only had one En insertion. The flanking sequences surrounding the En insertions in max4.1 and max4.2 were isolated by inverse PCR (IPCR). The IPCR method was performed essentially as described by Silver (1991). max4.1 and max4.2 genomic DNA was digested with NspI which cuts once in the 3′ end of En and the resulting fragments circularised by religation. The DNA was linearised with BssHII before PCR using outwardly facing primers specific for either the 5′ or 3′ ends of En:-5′ end primers:—

| | |
|---|---|
| SPM546 | 5′ CAGCCTCACTTAGCGTAAGC 3′ |
| SPM145 | 5′ ATTAAAAGCGTCGGTTTCATCGGGAC 3′ |

3′ end primers:—

| | |
|---|---|
| SPM8225 | 5′ TCGGCTTATTTCAGTAAGAGTG 3′ |
| SPM7650 | 5′ CTAGCATGATGTGAGCCTGAAC 3′ |

The PCRed IPCR products were cloned into the TA vector (Invitrogen) and sequenced. DNA database searches revealed that the flanking plant sequences were identical to regions in a sequenced A. thaliana (ecotype Columbia) BAC AL049915. This BAC was sequenced by The Sanger Centre, Cambridge as part of the EU Arabidopsis sequencing project. Sequence analysis shows that the En elements in max4.1 and max4.2 have inserted 433 bp apart, immediately after the MAX4 ‘ATG’ and in the first intron of MAX4 respectively (FIG. 2). The fact that both the En elements lie so close together provides strong evidence that the En elements have inserted into MAX4. Translation of putative open reading frames (ORFs) in the region identified a protein sequence from an ORF that has homology to the protein sequences of Ambystoma tigrinum RPE65 (Retinal Pigment Epithelial 65 Kd protein, Hamel et al., (1992)), A. thaliana and Zea Mays (Tan et al., (1997)) NCE (Neoxanthin Cleavage Enzyme) and Synechocytis and Pseudomonas paucimobilis LSD (Lignostilbene Dioxygenase). This homology, together with the identification of putative splice sites, enabled the MAX4 sequence to be deduced from the BAC sequence (FIG. 2). This assignment was confirmed by the isolation of a MAX4 cDNA (FIG. 3). [The BAC containing MAX4 has been annotated by MIPS (www.mips.biochem.mpg.de/proj/thal), and MAX4 has been identified as gene T16118.20—however the last exon of MAX4 has been incorrectly assigned. This results in the C-terminal sequence of the putative ORF being incorrect.] The MAX4 cDNA was obtained by PCR from cDNA made from RNA isolated from A. thaliana leaf axil regions. The primers were designed from the MAX4 genomic sequence and are shown in FIG. 2 and below:—

| | |
|---|---|
| 5′ ATGGCTTCTTTGATCACAACC 3′ | 1Forward |
| 5′ TTAATCTTTGGGGATCCAGC 3′ | 2952reverse |

Final confirmation that MAX4 was cloned was obtained by complementation of max4.1 and max4.2 by retransformation with a region of the AL049915 BAC 5-encompassing the putative MAX4 region. An 8928 bp XbaI fragment was subcloned from the AL049915 BAC into the XbaI site of the binary vector pCAMBIA 1300 (www.cambia.org.au) forming the plasmid pMAX4XbaI. MAX4 mutants were transformed using an agrobacterial transformation method basically as described in (Bechtold et al., (1993)) using Agrobacterial strain pGV3850 containing pMAX4XbaI. A significant proportion of the kanamycin resistant transformants had a wild-type phenotype. Thus pMAX4XbaI contains the MAX4 gene.

Complete sequencing of the MAX4 cDNA revealed that the cDNA was shorter than that shown in FIG. 3, the sequence from nucleotides 1467 to 1545 being absent. The complete MAX4 cDNA sequence is shown in FIG. 6. Sequencing revealed the presence of an additional intron within exon 4 of the MAX4 gene sequence (the new intron being between nucleotides 6146 and 6224 of FIG. 2). This finding resulted in a reduction in the size of the deduced MAX4 protein sequence from 596 amino acids to 570 amino acids with the loss of the internal 26 amino acid sequence TYIPQTIGFQYSIVLNEPFDNCMRQV. The revised deduced MAX4 protein sequences are now shown in FIG. 5 and FIG. 6.

### EXAMPLE 2

### Characterisation of MAX4

The homology of the putative MAX4 protein (unrevised sequence shown in FIG. 2 and FIG. 3) to RPE65, NCE and LSD is shown in FIG. 4. The homology of the putative MAX4 protein (revised sequence shown in FIG. 5 and FIG. 6) to RPE65, NCE and LSD is shown in FIG. 7. All these related sequences have blocks of similarity around conserved histidines (FIG. 4 and FIG. 7). Both NCE and LSD are thought to be dioxygenases involved in abscisic acid (ABA) and vanillin synthesis respectively. The chemical reactions catalysed by NCE and LSD are proposed to be very similar involving O₂ cleavage of 9-cis-carotenoid to xanthoxin in the case of NCE and ligostilbene to 2-vanillin in the case of LSD (Tan et al., (1997); FIG. 8). In dioxygenases of known structure conserved histidines are typical ligands of a non-haem iron cofactor, LSD being known to require non-haem iron for activity (Kanoda and Saburi (1993)). However MAX4 shows greatest homology to RPE65 which is required for the isomerization of all-trans-retinyl ester to 11-cis retinol (Redmond (1998)) and to recently identified beta-carotene 15, 15′-dioxygenases (beta-CD (BCDO)) which catalyse cleavage of beta-carotene forming all trans retinal (Redmond et al., (2001)) (see FIG. 4 and FIG. 7). Since these are mammalian rather than plant or cyanobacterial proteins, RPE65 and beta-CD are likely to catalyse a reaction closer to that catalysed by MAX4.

The reaction catalysed by RPE65 is similar to that proposed in ABA biosynthesis where isomerization of all-trans carotenoid precursors is a prerequisite for the subsequent oxidative cleavage catalysed by NCE (Tan et al., (1997); FIG. 9). There is evidence to implicate ABA in the transduction of the auxin-mediated apical dominance response. Auxin acts to control axillary bud outgrowth via a second messenger (Emery et al., (1998) and IAA, the natural plant auxin, may inhibit bud elongation by stimulating ABA biosynthesis in the bud (Tames et al., (1979). Supporting evidence comes from the following findings:—

a) ABA concentration in Xanthium buds increases after addition of exogenous auxins (Eliasson, (1974))

b) After release of apical dominance by decapitation of Phaseolus vulgaris the timing of lateral bud elongation correlated with a decrease in ABA level and could be reversed by IAA application (Knqx and Waring, (1984))

c) exogenous application of ABA to lateral buds inhibited elongation (Tames et al., (1979)).

Alternatively, MAX4 could cleave a carotenoid resulting in the formation of compounds that inhibit lateral branch elongation. These compounds could be ABA-like.

The expression pattern of MAX4 was initially investigated by RT PCR using primers specific for MAX4. First strand cDNA was made using primer OG1 and PCR performed using the MAX specific primers 2925R and 1F.

| | |
|---|---|
| 5′ GAGAGAGGATCCGGAGTTTTTTTTTTTTTTTT 3 | OG1 |
| 5′ ATGGCTTCTTTGATCACAACC 3 | 1Forward |
| 5′ TTAATCTTTGGGGATCCAGC 3 | 2952Reverse |

Preliminary results show that MAX4 transcript is only significantly present in mRNA isolated from the axils and lateral buds of A. thaliana. In these preliminary studies, no or insignificant expression could be observed in roots, mature leaves, internodes, flowers and siliques.

Analysis of the MAX4 protein sequence suggests that it contains a putative chloroplast transit peptide since it contains the transit peptide consensus sequence F/W-G/P-K/R (Pilon et al., (1995). It is known that ABA biosynthesis occurs in the chloroplast since chloroplast import of ABA2 (Zeanthin epoxidase) has been demonstrated (Marin et al., (1996)) and NCE also contains a putative chloroplast transit peptide. It is likely that MAX4 is a protein implicated in ABA biosynthesis. MAX4 may possibly be an axil specific protein.

### EXAMPLE 3

### Isolation and Characterisation of the MAX4 Promoter in A. thaliana and B. nanus

The primers BAC H −3578F and BAC B 17R were used to PCR a 3595 bp MAX4 promoter region from A. thaliana genomic DNA using TAQ DNA polymerase (Promega) (see FIGS. 2 and 5).

| | |
|---|---|
| 5′ TATAAGCTTGCTTGCTTTGTGGGGAAAC 3′ | BAC H-3578F |
| 5′ TTAGGATCCGTGATCAAAGAAGCCATC 3′ | BAG B 17R |
| BamHI | |

In earlier studies, the PCR fragment was cloned into pCR TOPO, using the Invitrogen TA system, and sequenced. The pMAX4 fragment was then excised as a BstXI, BamHI fragment from the pCR TOPO derivative and cloned as a BstXI, BamHI fragment into BstXI, BglII cut pCAMBIA 1381Xa (www.cambia.org.au) forming a translational fusion of MAX4 to GUS (FIG. 10c). The resulting plasmid, pMAX4GUS-CAMBIA, was then transferred into Agrobactenal strain pGV3850 and transformed into A. thaliana using the floral infiltration method. pMAX4-GUSCAMBIA was also transferred into Agrobacterial strain C58 pMP90 and transformed into B. napus essentially as described in Moloney M et al., (1989). GUS expression in both A. thaliana and B. napus transformants is restricted to leaf axils.

In subsequent studies, the PCR fragment was digested with EcoRI and BamHI and cloned between the EcoRI and BglII sites of pCAMBRIA 1303 (www.cambria.orp.au) forming a translational fusion of MAX4 to GUS (FIG. 10a). The resulting plasmid, pMAX4-GUS-CAMBIA, was then transferred into Agrobacterial strain pGV3850 and transformed into A. thaliana using the floral infiltration method. pMAX4-GUS-CAMBIA was also transferred into Agrobacterial strain C58 pMP90 and transformed into B. napus essentially as described in Moloney M et al., (1989). GUS expression in both A. thaliana and B. napus transformants is shown in FIG. 10a As can be seen in FIG. 10a, GUS expression was predominantly in the vasculature of leaves, stems, sepals, siliques and roots (replica transformed plants revealed a similar pattern of GUS expression). This expression may be in the phloem and/or xylem.

To produce a clean translational fusion of pMAX4 to GUS and other genes the primers pMAX4F and pMAX4R were used to PCR a 3578 bp MAX4 upstream DNA fragment from A. thaliana genomic DNA using proof-reading Tli polymerase (Promega) (see FIG. 2 and FIG. 5):—

| | |
|---|---|
| 5′ CTCTAGAGTTTTCTAAATGGACGATG 3′ | pMAX4F |
| XbaI | |
| 5′ GCCATGGTGGCAGAGTTTTTTTCTTTTC 3′ | pMAX4R |
| NcoI | |

The pMAX4F primer introduces an XbaI site at the 5′ end of the pMAX4 promoter fragment and the pMAX4R primer an NcoI site around the initiating ATG of MAX4. The PCR fragment was cloned into the SmaI site of pTZ18 (Pharmacia) and sequenced. The pMAX4 fragment was then cloned as an XbaI, NcoI fragment into XbaI, NcoI-cut pDH68 (WO99/13089) forming pMAX4-GUS. The pMAX4-GUS-CaMVpolyA region was then excised from pMAX4-GUS as an XbaI, XhoI fragment and cloned between the XbaI and SalI sites of the binary vector pNos-NptII-SCV (WO96/30529) forming pMAX4-GUS-SCV (FIG. 10b). This plasmid was then transferred into Agrobacterial strain pGV3580 and transformed into A. thaliana using the floral infiltration method. pMAX4-GUS-SCV was also transferred into Agrobacterial strain C58 pMP90 and transformed into B. napus essentially as described in Moloney M et al., (1989). GUS expression in both A. thaliana and B. napus transformants is as for pMAX4-GUS-CAMBRIA.

### EXAMPLE 4

### Increased in Aerial Branching in B. napus by Transformation with pMAX4-asMAX4 Constructs

An increase in aerial branching in plants can be achieved by downregulation of MAX4 expression or the orthologue of MAX4 in that plant species. MAX4 downregulation can be achieved by methods well known in the art, such as the expression of antisense, fill sense, partial sense transcripts homologous to MAX4 and the expression of ribozymes that are designed to cleave MAX4 transcipt. Additionally, given the sequence of MAX4, mutations in MAX4 can be readily identified in plant populations enabling the combination of mutant MAX allelles to provide partial of fill downregulation of MAX4 activity. Transcripts homologous to MAX4 or ribozymes may be expressed from any promoter that is expressed where MAX4 is expressed. Thus ‘constitutive’ promoters, such as the CaMV35 promoter, can be used. Axil-specific, leaf axil specific or vasculature specific promoters may be used. Preferably the promoter to be used is pMAX4.

To downregulate MAX4 expression in B. napus the A. thaliana MAX4 promoter is linked to an antisense fragment of the A. thaliana MAX4 coding region. The primers asMAX4F and asMAX4R are used to PCR a 1263 bp fragment from the MAX4 cDNA using non-proof-reading TAQ polymerase.

| | |
|---|---|
| 5′ GGGATCCAGGATGGCTTCTTTG 3′ | asMAX4F |
| BamHI | |
| 5′ ACCATGGGTTGAACGTAGGGTATCG 3′ | asMAX4R |
| NcoI | |

The primer asMAX4F introduces a BamHI site into the 3′ end of the antisense MAX4 PCR fragment. The asMAX4R fragment introduces base changes that create a stop codon downstream of the initiating ATG of the antisense MAX4 PCR fragment, thus preventing the antisense MAX4 expressing a peptide. The PCRed antisense MAX4 fragment is cloned into pGEM-T (Promega), then exised as an NcoI, BamH fragment and cloned between the NcoI and BamHI sites of pMAX4-GUS forming pMAX4-asMAX4. The pMAX4-asMAX4-CaMVpolyA region is then excised from pMAX4-asMAX4 as an XbaI, XhoI fragment and cloned between the XbaI and SalI sites of the binary vector pNos-NptlI-SCV forming pMAX4-asMAX4-SCV (FIG. 11). This plasmid is then transferred into Agrobacterial strain C58 pMP90 and transformed into B. napus. A proportion of transformed plants exhibit increased aerial branching leading to a slightly dwarfed bushy plants with more synchronous flowering than in wild-type plants.

The frequency and effectiveness of MAX4 downregulation in B. napus can be increased by substition of the A. thaliana antisense MAX4 fragment with that from B. napus MAX4. A B. napus orthologue of MAX4 (BnMAX4) is obtained by screening a B. napus cDNA library with MAX4 cDNA. PCR is used to introduce BamHI and NcoI into the ends of the BDMAX4 fragment PCRed from the BnMAX4 cDNA. The fragment is cloned in an antisense orientation behind the A. thaliana MAX4 promoter. A greater proportion of B. napus plants transformed with this pMAX4-asBnMAX4 construct exhibit increased aerial branching, dwarfing and synchronous flowering.

### EXAMPLE 5

### Decreased Aerial Branching by Transformation with a pMAX4-MAX4 Construct

Decreased aerial branching can have economic value for example in producing timber with fewer knots. Overexpression of MAX4 from a plant specific promoter, for example, an axil specific or vasculature specific promoter, may lead-to reduced lateral bud outgrowth with limited pleiotrophic effects. To exemplify this approach plants are transformed with MAX4. The Max4 cDNA is PCRed using the primers:—

| | |
|---|---|
| 5′ TCCATGGCTTCTTTGATCACAACC 3′ | sMAX4F |
| NcoI | |
| 5′ GTAGTTAATCTTTGGGGATC 3′ | sMAX4R |

The 1800 bp PCR product is cloned into SmaI-cut pTZ18 forming pMAX4s. The Max4 coding region is excised from pMAX4s as a partial NcoI, BamHI fragment and cloned between the NcoI and BamHI sites of pMAX4-GUS forming pMAX4-sMAX4. The pMAX4-sMAX4-CaMVpolyA chimeric gene is then cloned as an XbaI, XhoI fragment between the XbaI and SalI sites of the binary plasmid pNos-NptII-SCV (FIG. 12). This construct is transformed into agrobacteria and used to transform A. thaliana and B. napus. A proportion of transformed A. thaliana and B. napus plants exhibit reduced lateral bud outgrowth and are taller than wild-type plants.

### EXAMPLE 6

### Increase Resistance to Drought Stress by Expression of MAX4 in Leaves

MAX4 encodes a critical rate limiting step in ABA biosynthesis, thus overexpression of MAX4 from an appropriate promoter can phenocopy the effects of natural ABA overproduction. For example MAX4 overexpression from an embryo and/or endosperm-specific promoter can reduce preharvest sprouting, expression of MAX 4 in a bud-specific promoter can increase plant dormancy and expression of MAX4 in leaves or more preferably specifically stomatal cells can reduce stomatal aperture and thus increase plant drought tolerance. To exemplify this approach MAX4 is expressed from the pea plastocyanin promoter (Pwee K-H and Grey JC (1990)) which is expressed in green tissues and stomatal cells. The Max4 coding region is cloned as a partial NcoI, BamHI fragment from pMAX4s between the NcoI, BamHI sites of pDH68 forming pPcPea-sMAX4. The pPeaPC-sMAX4-CaMVpolyA chimeric gene is then cloned as an XbaI, XhoI fragment between the XbaI and SalI sites of the binary plasmid pNos-NptII-SCV (FIG. 13). This construct is transformed into agrobacteria and used to transform A. thaliana and B. napus. Detached leaves were measured for rate of water loss. A proportion of transformed A. thaliana and B. napus plants exhibit reduced water loss compared to untransformed control plants.

### REFERENCES

Ausebel et al., (1989) In current protocols in Molecular Biology. Green Publishing Associates, Inc, and John Wiley and Sons, Inc, New York. 1, 2.10.3.

Bechtold et al., (1993) C. R. Acad. Sci. Paris, Scila Vie/Life Sci. 316, 1194-1199.

Deveraux et al, (1984), Nucleic Acids Research 12 387.

Doebley et al, (1997). Nature. 386, 485-488.

Eliasson, L. (1974). Physiologa Plantarum. 34,117-120.

Emery, R., Longnecker, N., Atkis, C. (1998). J. Experiment Biology 49(320), 555-562.

Gubic V. and Bleecker A. B. (1996) Development 122 2395-2403.

Hamel, C., Tsilo, E., Pfeffer, B., Hooks, J., Detrick, B., Redmond, T. (1993). J. Biological Chemistry 268(21),15751-15757.

Iltis H. (1983) Science 222 886-894.

Kamoda, S and Saburi, Y. (1993). Biosci. Biotech. Biochem. 57, 926-930.

Knox, J. and Wareing, P. (1984). J. Experimental Biology 35(151), 239-244.

Mazin, E., Nussaume, L., Quesada, A., Gonneau, M., Sotta, B., Hugueney, P., Frey, A., Marion-Poll, A. (1996). EMBO J. 15(10),2331-2342.

Moloney M et al., (1989) Plant Cell Reports 8, 238-242.

Murray, M., Thompson, W. (1980). Rapid isolation of high molecular weight DNA. Nucleic Acids Research 8(19).

Needleman and Wunsch (1970), J. Mol. Biol. 48 443.

Pilon, M., Weink, H., Sips, W., Swaaf, m., Talboom, I., Van T Hof, R, Zorrilla, L., Bouffant, A., Korte-Kool, G., Demel, R., Weisbeek, P., Kruijff, B. (1994). J. Biological Chemistry 270(8), 3882-3893.

Pwee K-H and Grey JC (1990). Plant Journal 3, 4370-4449.

Redmond, T., Yu, S., Lee, L., Bok, D., Hamasaki, D., Chen, N., Goletz, P., Jian-Xing, M., Crouch, R., Pfeifer, K. (1998). Nature Genetics 20, 344-351.

Redmond, TM, Gentleman S, Duncan T, Yu S, Wiggert B, Gantt E and Cunningham FX (2001) Journal of Biological Chemistry 276, 6560-6565.

Schumacher K., Schmitt T., Rossberg M., Schmitz G., Theves K. (1999) Proc. Natl. Acad. Sci. USA 96 290-295.

Silver (1994). Inverse polymerase chain reaction. PCR a practical approach. Mcpherson, Quirke and Taylor. 9, 137-146.

Smith and Waterman (1981) Adv. Appl. Math 2 482.

Steeves T. A., Sussex I. M. (1989). In Patterns in Plant Development, 2^{nd} Edn. Cambridge University Press. 124-146.

Tan, B., Schwartz, S., Zeevaart, J., McCarty, D. (1997). Proc. Natl. Acad. Sci. 94, 12235-12240.

Tames, I, Ozbun, J., Wallace, D., Powell, L., Engels, C. (1979). Plant Physiology 64, 615-619.

1. Nucleic acid selected from
(i) a DNA sequence comprising all or part of the DNA sequence of FIG. 5 or FIG. 6 or its complementary straud;
(ii) nucleic acid sequences hybridising to the DNA sequence of FIG. 5 or FIG. 6 or its complementary strand under stringent conditions;
(iii) nucleic acid sequences which would hybrise to the DNA sequence of FIG. 5 or FIG. 6 or its complementary strand but for the degeneracy of the genetic code.

2. Nucleic acid as claimed in claim 1 which encodes a protein involved in the synthesis of abscisic acid (ABA).

3. Nucleic acid as claimed in claim 2 wherein the protein is one or more of isomerase, epoxidase, dioxygenase, oxygenate oxidase, oxgenase, hydroxylase, cyclase, D-expoxydase, desaturase or synthase.

4. Nucleic acid as claimed in any one of claims 1 to 3 which encodes a protein involved in the regulation of aerial branching.

5. Nucleic acid as claimed in any one of claims 1 to 4 which comprise the sequence set out in FIG. 5 or FIG. 6 or a fragment thereof which is at least 15 nucleotides in length.

6. Nucleic acid as claimed in any one of claims 1 to 5 wherein expression of the nucleic acid sequence in plants reduces the degree of aerial branching.

7. Nucleic acid which is antisense to nucleic acid as claimed in any one of claims 1 to 6.

8. Nucleic acid as claimed in claim 7 wherein expression of the antisense in plants increases the degree of aerial branching.

9. Nucleic acid encoding the amino acid sequence of FIG. 6.

10. Nucleic acid as claimed in any one of claims 1 to 9 including a promoter or other regulatory sequence which controls expression of the nucleic acid.

11. Nucleic acid which is the naturally occurring promoter which controls expression of nucleic acid as claimed in any one of claims 1 to 10.

12. Nucleic acid as claimed in claim 11 wherein expression of the nucleic acid under the control of the naturally occurring promoter in plants suppresses aerial branching.

13. Nucleic acid according to any one of claims 10 to 12 wherein the promoter comprises all or part of the underlined sequence as set out in FIG. 5.

14. Promoter sequence selected from
(i) a DNA sequence comprising all or part of the DNA sequence underlined in FIG. 5 or its complementary strand; and
(ii) nucleic acid sequences hybridising to the DNA sequence underlined in FIG. 5 or its complementary strand under stringent conditions.

15. Promoter as claimed in claim 14 in combination with nucleic acid of any one of claims 1 to 9.

16. Promoter as, claimed in claim 14 or claim 15 in combination with a gene of interest.

17. Promoter as claimed in any one of claims 14 to 16 which is vasculature specific.

18. Promoter as claimed in any one of claims 14 to 17 which is xylem specific.

19. RNA encoded by nucleic acid as claimed in any one of claims 1 to 13 or promoter as claimed in any one of claims 14 to 18.

20. A protein which is the expression product of a nucleic acid as claimed in any one of claims 1 to 13 or an RNA as claimed in claim 19.

21. An antibody capable of binding to a protein as claimed in claim 20.

22. nucleic acid as claimed in any one of claims 1 to 13 which is in the form of a vector.

23. A cell comprising nucleic acid as claimed in claim 22.

24. A plant cell as claimed in claim 23.

25. A process for obtaining a cell as claimed in claim 23 or claim 24 comprising introducing nucleic acid as claimed in any one of claims 1 to 13 into said cell.

26. A plant or a part thereof comprising a cell as claimed in claim 23 or claim 24.

27. Propagating material or a seed comprising a cell as claimed in claim 23 or claim 24.

28. A process for obtaining a plant or plant part as claimed in claim 26 comprising obtaining a cell as claimed in claim 25 and growth thereof or obtain a plant, plant part or propagating material as claimed in claim 27 and growth thereof.

29. A protein which:
(i) comprises the amino acid sequence shown in FIG. 5 or FIG. 6; or
(ii) has one or more amino acid deletions, insertions or substitutions relative to a protein as defined in (i) above, and has at least 40% amino acid sequence identity therewith; or
(iii) a fragment of a protein as defined in (i) or (ii) above which is at least 10 amino acids long.

30. Nucleic acid which encodes a protein as claimed in claim 29.

31. A protein as claimed in claim 29 or claim 30 which comprises a transit peptide sequence.

32. A protein as claimed in any one of claims 29 or 31 which is isolated or recombinant.

33. A process for regulating/controlling aerial branching in a plant or part thereof, the process comprising obtaining a plant or part thereof as claimed in claim 26.

34. A process as claimed in claim 33 which involves the synthesis of abscisic acid.

35. A process as claimed in claim 33 or claim 34 which comprises obtaining a plant cell as claimed in claim 24 or part of a plant as claimed in claim 26 and deriving a plant therefrom.

36. A process as claimed in any one of claims 33 to 35 which comprises obtaining a propagating material or a seed as claimed in claim 27 and deriving a plant therefrom.

37. A process as claimed in claim 33 wherein aerial branching is regulated at the leaf axil.

38. Use of nucleic acid as claimed in any one of claims 1 to 13 for the regulation of aerial branching in plants.

39. Use of nucleic acid as claimed in any one of claims 1 to 13 for the synthesis of abscisic acid.

40. Use of a nucleic acid as claimed in any one of claims 1 to 13 to regulate plant responses to water stress.

41. Use of nucleic acid as claimed in any one of claims 1 to 13 to regulate preharvest sprouting.

42. Use of nucleic acid as claimed in any one of claims 1 to 13 as a probe.

43. Use of nucleic acid as claimed in any one of claims 1 to 13 in the production of a cell, tissue, plant part thereof or propagating material.

44. Nucleic acid comprising one or more of the primer sequences in FIG. 5.

45. Use of the nucleic acid as claimed in claim 44 as a PCR primer.

46. Use of a protein as claimed in claim 29, claim 31 or claim 32 as a probe.

47. A method for the regulation of aerial branching in plants, the method comprising the steps of
(i) transforming the plant with nucleic acid as claimed in claim 1;
(ii) expression of said nucleic acid in a plant under the control of a promoter.

48. A method for regulating the synthesis of abscisic acid in plants, the method comprising the steps of
(i) transforming the plant with nucleic acid as claimed in claim 1;
(ii) expression of said nucleic acid in a plant under the control of a promoter.


SEQUENCE LISTING (47 sequences)

SEQ ID NO: 1 - DNA, 6708 bp, Arabidopsis thaliana
  CDS (3590)..(3904)
ttaaatatggagcttgctttgtggggaaaccctgattttgtccgccacaagtgaccaaaaaaaataaaataatgttttttgaattttaatacattgtaggaatcaattggggcatgaagggacattaagcaatttaaaaagggcgttgaatacacagaacgagttctctaaattgcatctcatctcatccagacatccaccatgacgctttagtgatggcattttagggtttagtggttcatcgataatttttgtatttcagatgttttctagttttccaatcattaaaggtagcattggaccaaaagatataaaaaacgataattaagagttgaaatttgaaaatcctgaaattccattaaactaagaaaagatgctttccctttctttattatcttcgtacattgactaaacatattcgatatagtgactgtgactcatgtgttctatcacattgcatcagcaatataattatataaacatttaaacttgacaacgaatatctgtcaatctatgtgctggattagttcgattaattgacattgtttcaaaaaagcgcacaaagtgtaataagtacacgataaatttaatacatattttttgtgccaaattagtaagtcgaatacctattggtacttgtatatatctttcgctgacatctgcgaactgaaattgaaaatctgaatatatatattttttgtgcacaaaaaatctgtttattcatttttaaaaaaaatgctagacactagttgactatatcactacaaataatgaaaaataaacagtaaaaccgcttaaggtgctgagttgacatatcatatatgaacatgttttcacttagaaataaaaaaaaaagaaatggaattcaccggcattttaatgaagaagggaaataaccaataatgattgaaggaagtgattattcttaagactagagacacatatcagatgagacaaacaaatgtggcaaacatatggacccaaatcgcagttttatattacaaggaatctcttgttaattagctagttgtccacggagttttgtactatataattcataattcgaatatggaaaactaatatgatatgaagatttagatacttttcattttcccttttgaacttctttccattttcccaattgggtatatggactaggaagaagaataatagaaggccatgtactcaaaccaataacatatatgtatatacaaaagggtagaattacatagcgaccaagataaataaacgttattctcttcttttctttttgaggtaaaaaaaaaatgtatatacattaataatagagaaatattccgacatacttatgaacaatcatgaaaataccttggataatatatataaaccatttcttgttgtaatattcatattaaaaatctaaatagtactattagagatattccacattttttgcaaggatatgcgtacttgataagtatatgaattttaattagaaaagtacaatttcattaaataaacaatatggtaaaattttagttttgaaaaggttaatagtattttagatatttagtaaataattgaaatgtattagtcaaaacatttgagttttctaaatggacgatgatttgaataattgtctaatactaatgtaaagatagagaaagtatcttatgcaaaaatttataaacattaaaaaaatatttgtatgagaaacttcttgtcgaggaccattatgaaaaaaaaatgtagtaactagttattatataaaagaataataggcttaatcgcatattatttaaaataaaatttcagttgacgcaaaaaaataatagaaaaatatattaaaattaataaaaaaagttctctctatattctttaaaacatatagaataatttaaaagaaaattagttgtaaataaattctctaaaaaaactaattgagaaaataaatatatgtcagtatttctatatattgacggttatcaaaatgtaagacattttactgtcatacaaatagacagacatatatgcatgacatgtttcatacaaacattctatgattgtatttatgtttttatatgtaaacgttacgtgtagaacaacgtttaagataatggtcatatcaatcaacctttaaatagatgaatcaaaggtgcttcctttgtttgaacttctaaatccacacaaaaatacaagaaaagtcaaataatgtggtctttatcatatttgtttgctccgaaaatatagtgtcacttgaacaaaactcttgacaaactggcctatactggttttttttcttaaccaatttatccgttaattttcaatgccgtattgggttcacttcgaagcatttcctgtcagtcattataatccaccaataatgtcagtttctctctctcgcccaaaaacgaacgatcgaggtgaatataaatgaaacatgtacattaattaatatatttatatcaaagttttttccggctttgaagcttgaaaatactccatatattatatacttctttgtgctaatttgtcgacgctttggccattttcatctatcatcgaggattcacaatttttggtgcgtatcgtttacatgatcgaagaaatagatatctgtaggatattatgacaatgtaaattaaatatagtttttctttagtctgttgattatctttatgcttagtttaagaaatagtgtaaacgcttaatatactcactttgcatgacccatataaacagtgtaaacgctaaaatataactcttcatcacccatattgatttaccttaaaatcacattcaccattgcgtctgatggaagtaatatttaagtatagaatacacccatatatatgtatatttattcataatattatatcaatatatattcatatatacgcgttgacaaaacagaaattcattctaacacgtacgtttatttaactttgataatgctagagtattattgacaggtataccgtgtacatggaattaatgtttttactcgctgctaataaaatgtatcttaccaccaatttaagcgaacgtatatcgtattattttaacaatagtttcagcagctgcaggtttccaacgactaatcttttaaattttggaatagcgatcatattcatcatatatcgttaataatcatgaaccaaatatgatttggtcgccaaaatctataatatcatcgttagaacttgtcgctatttttaaacctatcattaacttaacataagacaaacaagacttggttcttggttacaaccactagtgattgtaaaacgaattatcttaactatgaaagaggtatcctacaaattaaaaccgcatcaaaacttaccgtcaaacttcttttaaataattatcgtcatttacgcgtatctaaaaaatgaatatattatatcaacttgaataagaatccatcatttgtaacaagatcccttaagcgagaccctataaatacatcacatgcgtagcaccaaacattcactaaaacttcatttactttcttgaacttgtcttgtgccctctttccaatacactgaaatccacttcaaaatatttatttaataaaaagaaaagaaaagaaaaaaactctgccaggatggcttctttgatcacaaccaaagcaatgatgagtcatcatcatgttttgtcgtcaactagaatcactactctttattccgacaattccatcggcgatcaacaaataaaaacaaaacctcaagtccctcaccggttatttgctcggaggatcttcggtgtaaccagagctgtaattaattcagcggcaccgtctccgttgccggagaaagagaaggtggaaggtgagagacggtgtcatgttgcgtggacaagtgtacaacaagagaattgggagggtgaacttactgtccaaggaaagatacccacttggctggtttgtctttttatcatttttctatattgctccaaatatataacttatagctatattcgtggaaattttatacaaatatgtatcgtgcacactaaacacactcacactggcacatgcatatgtatataatacaagacaaaccccattcatcatgcattatttatagttattctatatattacgtatacatttttctttccttatacatgatttcattactaagaaagagtaagagctatttgtccaaaaaaaaaaaaaaaaaagaaagaaagagtaagagcttaggttagtacaccatgttcgtatttatatatcaatatatgcagtgagaatcagaagaaatagagaacaaaggttcagattttaataaagaagatatgctagtttccaaaaagatatatatgcacatatatattattagtctaggttatatatgttctattatttctacaagttttgttttctctattatttttacaagactacaagttctaaactggtcataggcatggttggtacttctacaagttgtactcttgaccattgcaatctaaaatcagcatacaatcatgtttcataaaatacaccaacaatcatgtaccaaaaaatctattttttttagtttttgataaattaataatatttttactaaaacaaaactccataaacaaaatcatcaaaatttttaaaaaaagaagtaaaaatgagacaagaaaactaatgatttaaaatgacatgcagaatggtacgtacctaagaaacggtcctggtctatggaacattggagaccacgatttccggcatctcttcgacggctactccacactcgtcaagcttcaattcgatggcggtcgtatattcgccgcccaccgtctccttgaatccgacgcttacaaagccgccaagaaacacaataggctttgttaccgtgaattctccgagactccaaaatcggtgatcataaacaaaaaccctttctccgggatcggagaaatcgtcaggcttttctccggagagtctttaacggacaacgccaacaccggagtgatcaaactcggtgacgggcgggtcatgtgtctgacggagactcaaaaaggatcgattttagtcgaccatgagacgctagagacgatcgggaaatttgagtacgacgacgtattgtccgatcatatgatccaatcagcgcatccgatagtgacggagacggagatgtggacgttgataccggatttggttaaaccgggttatcgggtcgtgaggatggaagccgggtcgaataaaagagaggttgtggggcgggtgaggtgtcgaagtgggtcgtggggacccggttgggtccattcgtttgcggtgacggagaattatgttgtaataccggaaatgcccctgagatattcggtgaagaatcttcttagagctgagccgacgccactttacaagttcgagtggtgtccccaagacggagcttttattcatgtcatgtccaaactcaccggagaagtcgtaagtgatacttactttatacagtcaatggtctttcaaatttttaggtttttattggttatagagttatatatatagtactatatatagtagatcatagtttatggatggtttttctttagttaactataacaacaaaataataggattcttgataatgtatatcattggaaattaaccatgtaacagaatattgtttgatggtttttttggtcatttgatttgaatatacataaaaccataacgttatatatggttaaaggtggctagcgtggaggttccagcatacgtaacgtttcacttcataaacgcgtatgaagaagataaaaatggcgatggaaaagcgacggtcatcattgcagattgttgtgaacacaacgccgatactcggatactcgatatgctccgtctcgataccctacgttcttcccatggtcacgacgttttacccgatgctaggtaatgtatataagggttactactcaatactcatcacctacatttttcagttttgattatagctggattaatgtaatcttatgttaggatcgggagattcaggataccattggacgggagcaaatacgggaaactagagacagccgtggaggcagagaagcatgggagagcgatggatatgtgcagcatcaatcctttgtatttgggtcaaaaataccgttacgtttatgcatgcggtgctcaacgaccttgtaacttccccaatgctctctccaaggtaacttacataccccaaactatcggtttccaatattcaatcgttttgaatgaaccttttgataattgtatgagacaggttgatattgtggagaagaaagtgaagaactggcacgagcatggtatgataccatctgaaccattcttcgtgcctcgacccggtgcaacccatgaggatgatggttagttaaaaattctttagatcttattggtctttcttcgacgtgagtttaatagttttggttttgtgaaggagtggtgatatcgatagtaagtgaagaaaatggaggaagctttgcaatcttgcttgatgggagctcctttgaagaaatagcaagagccaagtttccctatggccttccttatggcttgcatggttgctggatccccaaagattaactacaaagtctcaacaaagataccttcattatacaaaacacaacatatgtataattaataccctctgtgcaggttttgtaaattgttgtcccttatatatgctttttgtctatatatgtgatgtacaaaaccaaaataaaaggaacggattgtggtggatacagtta

SEQ ID NO: 2 - protein, 596 aa, Arabidopsis thaliana
MASLITTKAMMSHHHVLSSTRITTLYSDNSIGDQQIKTKPQVPHRLFARRIFGVTRAVINSAAPSPLPEKEKVEGERRCHVAWTSVQQENWEGELTVQGKIPTWLNGTYLRNGPGLWNIGDHDFRHLFDGYSTLVKLQFDGGRIFAAHRLLESDAYKAAKKHNRLCYREFSETPKSVIINKNPFSGIGEIVRLFSGESLTDNANTGVIKLGDGRVMCLTETQKGSILVDHETLETIGKFEYDDVLSDHMIQSAHPIVTETEMWTLIPDLVKPGYRVVRMEAGSNKREVVGRVRCRSGSWGPGWVHSFAVTENYVVIPEMPLRYSVKNLLRAEPTPLYKFEWCPQDGAFIHVMSKLTGEVVASVEVPAYVTFHFINAYEEDKNGDGKATVIIADCCEHNADTRILDMLRLDTLRSSHGHDVLPDARIGRFRIPLDGSKYGKLETAVEAEKHGRAMDMCSINPLYLGQKYRYVYACGAQRPCNFPNALSKVTYIPQTIGFQYSIVLNEPFDNCMRQVDIVEKKVKNWHEHGMIPSEPFFVPRPGATHEDDGVVISIVSEENGGSFAILLDGSSFEEIARAKFPYGLPYGLHGCWIPKD

SEQ ID NO: 3 - DNA, 20 bp, Artificial sequence
  Primer
gctccatccccaaagattaa

SEQ ID NO: 4 - DNA, 28 bp, Artificial sequence
  Primer
tataagcttgcttgctttgtggggaaac

SEQ ID NO: 5 - DNA, 26 bp, Artificial sequence
  Primer
ctctagagcttgctttgtggggaaac

SEQ ID NO: 6 - DNA, 29 bp, Artificial sequence
  Primer
gaaaagaaaaaaactctgccaccatggcg

SEQ ID NO: 7 - DNA, 27 bp, Artificial sequence
  Primer
gatggcttctttgatcacggatcctaa

SEQ ID NO: 8 - DNA, 21 bp, Artificial sequence
  Primer
atggcttctttgatcacaacc

SEQ ID NO: 9 - DNA, 6708 bp, Arabidopsis thaliana; identical to SEQ ID NO: 1
  CDS (3590)..(3904)

SEQ ID NO: 10 - protein, 570 aa, Arabidopsis thaliana [sequence omitted]

SEQ ID NO: 11 - protein, 570 aa, Arabidopsis thaliana [sequence omitted]

SEQ ID NO: 12 - protein, 533 aa, Gallus gallus
MYSQVEHPAGGYKKLFETVEELSSPVTAHVTGRIPTWLRGSLLRCGPGLFEVGAEPFYHLFDGQALLHKFDFKEGHVTYHRRFVRTDAYVRAMTEKRIVITEFGTYAYPDPCKNIFSRFFSYFKGVEVTDNALVNVYPVGEDYYACTETNFITKINPDTLETIKQVDLCKYVSVNGATAHPHVENDGTVYNIGNCFGKNFSLAYNIIRIPPLQADKEDPMNKSEVVVQFPCSDRFKPSYVHSFGLTPNYIVFVETPVKINLLKFLSSWSLWGANYMDCFESNETMGVWLHVAEKKKGRLLNIKYRTSAFNLFHHINTFEDNGFLIVDLCTWKGFEFVYNYLYLANLRANWDEVKKQAEKAPQPEARRYVLPLRIDKADTGKNLVTLPYTTATATLRSDETVWLEPEVIFSGPRHAFEFPQINYKKYGGKPYTYTYGLGLNHFVPDRLCKLNVKTKETWVWQEPDSYPSEPIFVSHPDALEEDDGVVLSIVISPGSGPKPAYLLILNAKDMSEVARAEVEVNIPVTFHGLFKRA

SEQ ID NO: 13 - protein, 533 aa, Arabidopsis tigrinum
MTNRVDHPAGGYKKLFESTEELVAPVTAQVTGRIPVWLSGSLLRCGPGLFEVGSEQFYHLFDGQALLHKFEFKGGHVIYHRRFIRTDTYVRAMTEKRIVITEFGTFAFPDPCKNIFSRFLSYFQGLEVTDNALVNVYPVGEDYYACTETNYITKINPETLETVKKVDLCNYVSINGVTAHPHIEHDGTVYNIGNCFGKHFAFAYNIVKIPPLQADKEDPINKAKVVVQFPCSERFKPSYVHSFGLTPNYIVFVEQPVKINLFKFLSSWSIWGANYMDCFESHETMGVWMHVAEKHTGEYLNIKYRTSAFNLFHHINTYEDHGFLIVDLCCWKGFEFVYNYLYLANLRENWEEVKRSAEKPPQPEVRRYVLPLDIHKVDTGKNLVNLPYTTATAVLRSDETIWLEPEVLFSGPRQAFEFPQINYKKHGGKDYTYAYGVGLNHFVPDRLSKLNVKTKETWVWQEPDTYPSEPIFVSQPDAIEEDDGVVLSVVISPGEGQKPAFLLILNAKDMSEIARAEVDSNIPVTFHGMFKKA

SEQ ID NO: 14 - protein, 615 aa, Unknown
  Bean
MPSPASNTWINTTLPSSCSSPFKDLASTSSSPTTLLPFKKRSSSNTNTITCSLQTLHYPKQYQPTSTSTTTTPTPIKPTTTTTTTTPHRETKPLSDTKQPFPQKWNFLQKAAATGLDMVETALVSHESKHPLPKTADPKVQIAGNFAPVPEHAADQALPVVGKIPKCIDGVYVRNGANPLYEPVAGHHFFDGDGMVHAVKFTNGAASYACRFTETQRLAQEKSLGRPVFPKAIGELHGHSGIARLLLFYARSLFQLVDGSHGMGVANAGLVYFNNHLLAMSEDDLPYHVRITSNGDLTTVGRYDFNGQLNSTMIAHPKLDPVNGDLHALSYDVVQKPYLKYFRFSADGVKSPDVEIPLKEPTMMHDFAITENFVVVPDQQVVFKLTEMITGGSPVVYDKNKTSRFGILDKNAKDANAMRWIDAPECFCFHLWNAWEEPETDEIVVIGSCMTPADSIFNECDESLKSVLSEIRLNLRTGKSTRRPIISDAEQVNLEAGMVNRNKLGRKTQFAYLALAEPWPKVSGFAKVDLFSGEVQKYMYGEEKFGGEPLFLPNGEEEGDGYILAFVHDEKEWKSELQIVNAQNLKLEASIKLPSRVPYGFHGTFIHSKDLRKQA

SEQ ID NO: 15 - protein, 538 aa, Arabidopsis thaliana
MAEKLSDGSIIISVHPRPSKGFSSKLLDLLERLVVKLMHDASLPLHYLSGNFAPIRDETPPVKDLPVHGFLPECLNGEFVRVGPNPKFDAVAGYHWFDGDGMIHGVRIKDGKATYVSRYVKTSRLKQEEFFGAAKFMKIGDLKGFFGLLMVNIQQLRTKLKILDNTYGNGTANTALVYHHGKLLALQEADKPYVIKVLEDGDLQTLGIIDYDKRLTHSFTAHPKVDPVTGEMFTFGYSHTPPYLTYRVISKDGIMHDPVPITISEPIMMHDFAITETYAIFMDLPMHFRPKEMVKEKKMIYSFDPTKKARFGVLPRYAKDELMIRWFELPNCFIFHNANAWEEEDEVVLITCRLENPDLDMVSGKVKEKLENFGNELYEMRFNMKTGSASQKKLSASAVDFPRINECYTGKKQRYVYGTILDSIAKVTGIIKFDLHAEAETGKRMLEVGGNIKGIYDLGEGRYGSEAIYVPRETAEEDDGYLIFFVHDENTGKSCVTVIDAKTMSAEPVAVVELPHRVPYGFHALFVTEEQLQEQTLI

SEQ ID NO: 16 - protein, 490 aa, Synechocystis sp.
MVTSPPTSSPSQRSYSPQDWLRGYQSQPQEWDYWVEDVEGSIPPDLQGTLYRNGPGLLEIGDRPLKHPFDGDGMVTAFKFPGDGRVHFQSKFVRTQGYVEEQKAGKMIYRGVFGSQPAGGWLKTIFDLRLKNIANTNITYWGDRLLALWEGGQPHRLEPSNLATIGLDDLGGILAEGQPLSAHPRIDPASTFDGGQPCYVTFSIKSSLSSTLTLLELDPQGKLLRQKTETFPGFAFIHDFAITPHYAIFLQNNVTLNGLPYLFGLRGAGECVQFHPDKPAQIILVPRDGGEIKRIPVQAGFVFHHANAFEENGKIILDSICYNSLPQVDTDGDFRSTNFDNLDPGQLWRFTIDPAAATVEKQLMVSRCCEFPVVHPQQVGRPYRYVYMGAAHHSTGNAPLQAILKVDLESGTETLRSFAPHGFAGEPIFVPRPGGVAEDDGWLLCLIYKADLHRSELVILDAQDITAPAIATLKLKHHIPYPLHGSWAQT

SEQ ID NO: 17 - protein, 485 aa, Pseudomonas sp.
MAHFPQTPGFSGTLRPLRIEGDILDIEIEGEVPPQLNGTFHRVHPDAQFPPRFEDDQFFNGDGMVSLFRFHDGKIDFRQRYAQTDKWKVERKAGKSLFGAYRNPLTDDASVQGMIRGTANTNVMVHAGKLYAMKEDSPCLIMDPLTLETEGYTNFDGKLQSQTFCAHPKIDPVTGNLCAFAYGAKGLMTLDMAYIEISPTGKLLKEIPFQNPYYCMMHDFGVTEDYAVFAVMPLLSSWDRLEQRLPFFGFDTTLPCYLGILPRNGDARDLRWFKTGNCFVGHVMNAFNDGTKVHIDMPVSRNNSFPFFDVHGAPFDPVAGQGFLTRWTVDMASNGDSFEKTERLFDRPDEFPRIDERYATRAYRHGWMLILDTEKPYEAPGGAFYALTNTLGHIDLATGKSSSWWAGPRCAIQEPCFIPRSPDAPEGDGYVIALVDDHVANYSDLAIFDAQHVDQGPIARAKLPVRIRQGLHGNWADASRLAVAA

SEQ ID NO: 18 - protein, 526 aa, Gallus gallus
METIFNRNKEEHPEPIKAEVQGQLPTWLQGVLLRNGPGMHTIGDTKYNHWFDGLALLHSFTFKNGEVYYRSKYLRSDTYNCNIEANRIVVSEFGTMAYPDPCKNIFAKAFSYLSHTIPEFTDNCLINIMKTGDDYYATSETNFIRKIDPQTLETLDKVDYSKYVAVNLATSHPHYDSAGNILNMGTSIVDKGRTKYVLFKIPSSVPEKEKKKSCFKHLEVVCSIPSRSLLQPSYYHSFGITENYIVFIEQPFKLDIVKLATAYIRGVNWASCLSFHKEDKTWFHFVDRKTKKEVSTKFYTDALVLYHHINAYEEDGHVVFDIVAYRDNSLYDMFYLKKLDKDFEVNNKLTSIPTCKRFVVPLQYDKDAEVGSNLVKLPTSATAVKEKDGSIYCQPEILCEGIELPRVNYDYNGKKYKYVYATEVQWSPVPTKIAKLNVQTKEVLHWGEDHCWPSEPIFVPSPDAREEDEGVVLTCVVVSEPNKAPFLLILDAKTFKELGRATVNVEMHLDLHGMFIPQNDLGAETE

SEQ ID NO: 19 - protein, 549 aa, Mus musculus
MEIIFGQNKKEQLEPVQAKVTGSIPAWLQGTLLRNGPGMHTVGESKYNHWFDGLALLHSFSIRDGEVFYRSKYLQSDTYIANIEANRIVVSEFGTMAYPDPCKNIFSKAFSYLSHTIPDFTDNCLINIMKCGEDFYATTETNYIRKIDPQTLETLEKVDYRKYVAVNLATSHPHYDEAGNVLNMGTSVVDKGRTKYVIFKIPATVPDSKKKGKSPVKHAEVFCSISSRSLLSPSYYHSFGVTENYVVFLEQPFKLDILKMATAYMRGVSWASCMSFDREDKTYIHIIDQRTRKPVPTKFYTDPMVVFHHVNAYEEDGCVLFDVIAYEDSSLYQLFYLANLNKDFEEKSRLTSVPTLRRFAVPLHVDKDAEVGSNLVKVSSTTATALKEKDGHVYCQPEVLYEGLELPRINYAYNGKPYRYIFAAEVQWSPVPTKILKYDILTKSSLKWSEESCWPAEPLFVPTPGAKDEDDGVILSAIVSTDPQKLPFLLILDAKSFTELARASVDADMHLDLHGLFIPDADWNAVKQTPAETQEVENSDHPTDPQHQN

SEQ ID NO: 20 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 21 - protein, 604 aa, Zea mays
MQGLAPPTSVSIHRHLPARSRARASNSVRFSPRAVSSVPPAECLQAPFHKPVADLPAPSRKPAAIAVPGHAAAPRKAEGGKKQLNLFQRAAAAALDAFEEGFVANVLERPHGLPSTADPAVQIAGNFAPVGERPPVHELPVSGRIPPFIDGVYARNGANPCFDPVAGHHLFDGDGMVHALRIRNGAAESYACRFTETARLRQERAIGRPVFPKAIGELHGHSGIARLALFYARAACGLVDPSAGTGVANAGLVYFNGRLLAMSEDDLPYHVRVADDGDLETVGRYDFDGQLGCAMIAHPKLDPATGELHALSYDVIKRPYLKYFYFRPDGTKSDDVEIPLEQPTMIHDFAITENFVVVPDHQVVFKLQEMLRGGSPVVLDKEKTSRFGVLPKHAADASEMAWVDVPDCFCFHLWNAWEDEATGEVVVIGSCMTPADSIFNESDERLESVLTEIRLDARTGRSTRRAVLPPSQQENLEVGMVNRNLLGRESRYAYLAVAEPWPKESGFAKEDLSTGELTKFEYGEGRFGGEPCFVPMDPAAAHPRGEDDGYVLTFVHDERAGTSELLVVNAADIRLEATVQLPSRVPFGFHGTFITGQELEAQAA

SEQ ID NO: 22 - protein, 533 aa, Canis familiaris
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEVVVQFPCSDRFKPSYVHSFGLTPNYIVFVETPVKINLLKFLSSWSLWGANYMDCFESNETMGVWLHIADKKRKKYLNNKYRTSSFNLFHHINTYEDNEFLIVDLCCWKGFEFVYNYLYLANLRENWEEVKKNARKAPQPEVRRYVLPLNIDKADTGKNLVTLPNTTATATLRSDETIWLEPEVLFSGPRQAFEFPQINYQKSGGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 23 - protein, 608 aa, Lycopersicon esculentum
NCEMATTTSHATNTWIKTKLSMPSSKEFGFASNSISLLKNQHNRQSLNINSSLQAPPILHFPKQSSNYQTPKNNTISHPKQENNNSSSSSTSKWNLVQKAAAMALDAVESALTKHELEHPLPKTADPRVQISGNFAPVPENPVCQSLPVTGKIPKCVQGVYVRNGANPLFEPTAGHHFFDGDGMVHAVQFKNGSASYACRFTETERLVQEKALGRPVFPKAIGELHGHSGIARLMLFYARGLFGLVDHSKGTGVANAGLVYFNNRLLAMSEDDLPYHVKVTPTGDLKTEGRFDFDGQLKSTMIAHPKLDPVSGELFALSYDVIQKPYLKYFRFSKNGEKSNDVEIPVEDPTMMHDFAITENFVVIPDQQVVFKMSEMIRGGSPVVYDKNKVSRFGILDKYAKDGSDLKWVEVPDCFCFHLWNAWEEAETDEIVVIGSCMTPPDSIFNECDEGLKSVLSEIRLNLKTGKSTRKSIIENPDEQVNLEAGMVNRNKLGRKTEYAYLAIAEPWPKVSGFAKVNLFTGEVEKFIYGDNKYGGEPLFLPRDPNSKEEDDGYILAFVHDEKEWKSELQIVNAMSLKLEATVKLPSRVPYGFHGTFINANDLANQA

SEQ ID NO: 24 - protein, 286 aa, Artificial sequence
  Consensus sequence
VPVAVGIPWLGSLLRGPGLVGFHFDGALFDFGVYRIRTDYIGNRIVEFGAFPDPCKNIFSRFTDNGLVVVKVGEYACTETNIIPTLETIDLRVSVNGTAHPLVDGVNIGYLAYIKIPLIKEVQLSPYHSFGLTNYIVVEPKLLKFSIYCTMWFHIELNVFRMVFHINYEDLIDLLVMYLLFHNARRYVIPLIKADTNVTALRDTVLGEFPINYGKYRYAYGLNPVPLKLDLTKEWWEYPSEPIFVPPAEEDDGVVLSVVESFLLILNAKLSEVARALIPFHGLFIA

SEQ ID NO: 25 - DNA, 20 bp, Artificial sequence
  Primer
cagcctcacttagcgtaagc

SEQ ID NO: 26 - DNA, 26 bp, Artificial sequence
  Primer
attaaaagcgtcggtttcatcgggac

SEQ ID NO: 27 - DNA, 22 bp, Artificial sequence
  Primer
tcggcttatttcagtaagagtg

SEQ ID NO: 28 - DNA, 22 bp, Artificial sequence
  Primer
ctagcatgatgtgagcctgaac

SEQ ID NO: 29 - DNA, 21 bp, Artificial sequence
  Primer
atggcttctttgatcacaacc

SEQ ID NO: 30 - DNA, 20 bp, Artificial sequence
  Primer
ttaatctttggggatccagc

SEQ ID NO: 31 - protein, 26 aa, Arabidopsis thaliana
TYIPQTIGFQYSIVLNEPFDNCMRQV

SEQ ID NO: 32 - DNA, 32 bp, Artificial sequence
  Primer
gagagaggatcccgagtttttttttttttttt

SEQ ID NO: 33 - DNA, 20 bp, Artificial sequence
  Primer
ttaatctttggggatccagc

SEQ ID NO: 34 - DNA, 28 bp, Artificial sequence
  Primer
tataagcttgcttgctttgtggggaaac

SEQ ID NO: 35 - DNA, 27 bp, Artificial sequence
  Primer
ttaggatccgtgatcaaagaagccatc

SEQ ID NO: 36 - DNA, 26 bp, Artificial sequence
  Primer
ctctagagttttctaaatggacgatg

SEQ ID NO: 37 - DNA, 28 bp, Artificial sequence
  Primer
gccatggtggcagagtttttttcttttc

SEQ ID NO: 38 - DNA, 22 bp, Artificial sequence
  Primer
gggatccaggatggcttctttg

SEQ ID NO: 39 - DNA, 25 bp, Artificial sequence
  Primer
accatgggttgaacgtagggtatcg

SEQ ID NO: 40 - DNA, 24 bp, Artificial sequence
  Primer
tccatggcttctttgatcacaacc

SEQ ID NO: 41 - DNA, 20 bp, Artificial sequence
  Primer
gtagttaatctttggggatc

SEQ ID NO: 42 - DNA, 1791 bp, Arabidopsis thaliana
  CDS (1)..(1788)
atggcttctttgatcacaaccaaagcaatgatgagtcatcatcatgttttgtcgtcaactagaatcactactctttattccgacaattccatcggcgatcaacaaataaaaacaaaacctcaagtccctcaccggttatttgctcggaggatcttcggtgtaaccagagctgtaattaattcagcggcaccgtctccgttgccggagaaagagaaggtggaaggtgagagacggtgtcatgttgcgtggacaagtgtacaacaagagaattgggagggtgaacttactgtccaaggaaagatacccacttggctgaatggtacgtacctaagaaacggtcctggtctatggaacattggagaccacgatttccggcatctcttcgacggctactccacactcgtcaagcttcaattcgatggcggtcgtatattcgccgcccaccgtctccttgaatccgacgcttacaaagccgccaagaaacacaataggctttgttaccgtgaattctccgagactccaaaatcggtgatcataaacaaaaaccctttctccgggatcggagaaatcgtcaggcttttctccggagagtctttaacggacaacgccaacaccggagtgatcaaactcggtgacgggcgggtcatgtgtctgacggagactcaaaaaggatcgattttagtcgaccatgagacgctagagacgatcgggaaatttgagtacgacgacgtattgtccgatcatatgatccaatcagcgcatccgatagtgacggagacggagatgtggacgttgataccggatttggttaaaccgggttatcgggtcgtgaggatggaagccgggtcgaataaaagagaggttgtggggcgggtgaggtgtcgaagtgggtcgtggggacccggttgggtccattcgtttgcggtgacggagaattatgttgtaataccggaaatgcccctgagatattcggtgaagaatcttcttagagctgagccgacgccactttacaagttcgagtggtgtccccaagacggagcttttattcatgtcatgtccaaactcaccggagaagtcgtggctagcgtggaggttccagcatacgtaacgtttcacttcataaacgcgtatgaagaagataaaaatggcgatggaaaagcgacggtcatcattgcagattgttgtgaacacaacgccgatactcggatactcgatatgctccgtctcgataccctacgttcttcccatggtcacgacgttttacccgatgctaggatcgggagattcaggataccattggacgggagcaaatacgggaaactagagacagccgtggaggcagagaagcatgggagagcgatggatatgtgcagcatcaatcctttgtatttgggtcaaaaataccgttacgtttatgcatgcggtgctcaacgaccttgtaacttccccaatgctctctccaaggtaacttacataccccaaactatcggtttccaatattcaatcgttttgaatgaaccttttgataattgtatgagacaggttgatattgtggagaagaaagtgaagaactggcacgagcatggtatgataccatctgaaccattcttcgtgcctcgacccggtgcaacccatgaggatgatggagtggtgatatcgatagtaagtgaagaaaatggaggaagctttgcaatcttgcttgatgggagctcctttgaagaaatagcaagagccaagtttccctatggccttccttatggcttgcatggttgctggatccccaaagattaa

SEQ ID NO: 43 - protein, 596 aa, Arabidopsis thaliana; identical to SEQ ID NO: 2

SEQ ID NO: 44 - protein, 10 aa, Artificial sequence
  MAX4 GUS fusion sequence
MASLITDLTS

SEQ ID NO: 45 - DNA, 33 bp, Artificial sequence
  MAX4 GUS fusion sequence
atggcttctttgatcacggatctgactagttta

SEQ ID NO: 46 - protein, 569 aa, Arabidopsis thaliana
MASLITTKAMMSHHHVLSSTRITTLYSDNSIGDQQIKTKPQVPHRLFARRIFGVTRAVINSAAPSPLPEKEKVEGERRCHVAWTSVQQENWEGELTVQGKIPTWLNGTYLRNGPGLWNIGDHDFRHLFDGYSTLVKLQFDGGRIFAAHRLLESDAYKAAKKHNRLCYREFSETPKSVIINKNPFSGIGEIVRLFSGESLTDNANTGVIKLGDGRVMCLTETQKGSILVDHETLETIGKFEYDDVLSDHMIQSAHPIVTETEMWTLIPDLVKPGYRVVRMEAGSNKREVVGRVRCRSGSWGPGWVHSFAVTENYVVIPEMPLRYSVKNLLRAEPTPLYKFEWCPQDGAFIHVMSKLTGEVVASVEVPAYVTFHFINAYEEDKNGDGKATVIIADCCEHNADTRILDMLRLDTLRSSHGHDVLPDARIGRFRIPLDGSKYGKLETAVEAEKHGRAMDMCSINPLYLGQKYRYVYACGAQRPCNFPNALSKVDIVEKKVKNWHEHGMIPSEPFFVPRPGATHEDDGVVISIVSEENGGSFAILLDGSSFEEIARAKFPYGLPYGLHGCWIPK

SEQ ID NO: 47 - protein, 286 aa, Artificial sequence; identical to SEQ ID NO: 24
  Consensus sequence